(19) United States
(12) Patent Application Publication
(10) Pub. No.: US 2005/0112626 A1
(43) Pub. Date: May 26, 2005

(54) Making and using microclonal uncloned cDNA libraries

(76) Inventors: Oleg N. Suslov, Gainesville, FL (US); Dennis A. Steindler, Gainesville, FL (US); Valery G. Kukekov, Gainesville, FL (US)

Correspondence Address: EDWARDS & ANGELL, LLP, P.O. BOX 55874, BOSTON, MA, 02205, US

(21) Appl. No.: 10/924,367

(22) Filed: Aug. 23, 2004

Related U.S. Application Data
(62) Division of application No. 09/527,785, filed on Mar. 17, 2000, now abandoned.
(60) Provisional application No. 60/124,897, filed on Mar. 17, 1999.

(51) Int. Cl. C12Q 1/68; C12P 19/34; C12N 5/08; C12N 15/85

(52) U.S. Cl. 435/6; 435/91.2; 435/368; 435/455

(57) ABSTRACT

A method is described for the preparation, characterization and utilization of temporally ordered panels of cDNA libraries from human microclones. Each microclone undergoing morphogenetic differentiation in vitro differs in discrete gene expression. The developmental stage of the microclone is determined and the gene expression patterns of the microclones temporally ordered and arranged into expression profiles and temporal spectra. Direct comparison of the cDNAs produced from different neural microclones allows identification of novel RNA transcripts.

### 1.0 BACKGROUND OF THE INVENTION

This application claims priority from provisional application U.S. Ser. No. 60/124,897, filed Mar. 17, 1999. The U.S. government has rights in this application through NIH/NIDS #NS29225.

1.1 Field of the Invention

The invention relates to the field of molecular biology and in particular to microclonal cDNA compositions and methods for making panels of temporally ordered gene expression products from stem/progenitor cells.

1.2 Description of Related Art

Neurogenesis in the mature mammalian brain has been a controversial issue for several years. Despite the work of Allen (1912) and others (Altman, 1969a; 1969b) supporting the existence of persistent neurogenesis in the adult rat olfactory and hippocampal systems, these were considered highly specialized cases that by no means supported a notion of neuropoiesis in the adult central nervous system. The in vitro propagation of an adult rat brain putative stem cell population (Reynolds and Weiss, 1992; Richards et al., 1992) suggested neuropoiesis; and later studies established the source of stem/progenitor cells as the subependymal zone, ependyma, and hippocampus (Johansson et al., 1999; Eriksson et al., 1998; Kukekov et al., 1999), regions recently amalgamated under one term—“brain marrow” (Steindler et al., 1996).

Extracellular matrix (ECM) and other developmentally-regulated molecules define a persistent neurogenic region of the adult mouse and human forebrain—the subependymal zone (SEZ) (Scheffler et al., 1999). The periventricular SEZ, as well as the ependymal layer has been referred to as “brain marrow” because of the similarity to hematopoietic bone marrow, where a central core of stem and precursor cells, surrounded by support cells and developmentally regulated molecules can give rise to a diversity of stem/progenitor cells (Steindler et al., 1996). “Stem/progenitor” is used to describe the full spectrum of proliferative cells that can give rise to all cells of a given tissue (Scheffler et al, 1999). Earlier studies of putative stem cells in the developing as well as mature rodent forebrain neuronal and glial progenitors were isolated in the presence of growth factors, including epidermal or fibroblast growth factors, EGF and FGF (Reynolds and Weiss, 1996; Gritti et al., 1996).

Culture methods for the isolation and characterization of pluripotent precursors from neural crest, fetal and embryonic nervous system are well-established (Calof et al., 1998), yet the in vitro generation of neurospheres and de novo-generated neurons from the adult mouse forebrain (Reynolds and Weiss, 1992; Richards et al., 1992) presented the first convincing description of pluripotent stem/progenitor cells in the mature CNS. It has been 80 years since the first documented observation of mitotic activity in the adult brain (Allen, 1912), and now there is strong evidence for a proliferative ancestor in the adult CNS that seems to possess all of the characteristics of a stem cell.

The analogy of a brain neuropoietic core to the hematopoietic bone marrow has been confirmed by the surprising finding that adult brain-derived stem/progenitor cells are pluripotent—giving rise to blood cells after homing to bone marrow following systemic grafting (Bjornson et al., 1999).

Because stem/progenitor cells can only be studied as clonal colony-like units or “neurospheres” (Scheffler et al., 1999), there has been a need for methods to isolate these cells in order to identify expressed molecules as well as factors that affect their growth and differentiation. Studies of neurospheres have focused on genetic analyses of populations of neurospheres, apparently because of difficulties in disrupting individual neurospheres. There is also difficulty in obtaining a significant amount of material from the mechanical or chemical disruption of neurospheres as well as the limited amount of information obtained from genetic material in the individual clones.

cDNA libraries and subtractive methods have been used to temporally order gene expression in species such as yeast, but not for neural gene expression. A previous study described a cluster analysis for gene expression from DNA microarray hybridization using “ . . . standard statistical algorithms to arrange genes according to similarity in pattern of gene expressions . . . ” This method has been primarily used to group genes in clusters according to known similar function (e.g., as in the case of studies on the budding yeast Saccharomyces cerevisiase as well as in the human) (Eisen et al., 1998).

A reverse transcriptase polymerase chain reaction (RT-PCR) has been applied to populations of neurospheres for the confirmation of cell phenotype- and growth factor-related molecules associated with these unique structures (Arsenijevic and Weiss, 1998). However, as recently emphasized, “ . . . . There are also basic technical issues relating to the growth and propagation of these cells in culture that need to be overcome. Specifically, the reported difficulty in dissociating the human embryonic stem and embryonic germ cell lines (ES/EG) cell clusters into viable single cells is problematic, particularly for gene-targeting experiments . . . ” (Keller and Snodgrass, 1999). These stem cell-generated cell structures, like neurospheres, thus pose a similar obstacle for gene discovery studies.

Currently, methods to generate cDNA libraries from cells undergoing specific cellular processes rely on the isolation of cells from organs or tissues at specific stages during that process. In order to study neurogenesis, cDNA libraries must be isolated from the neuronal cells of embryonic brains at various stages of embryonic brain development. The cDNA library obtained from an eleven-day-old embryonic mouse brain, for example, can be compared to the cDNA library obtained from a 17-day-old embryonic brain. Similarly, in order to study oncogenesis, cDNA libraries must be isolated from tumor cells at various stages of tumor development.

However, while these methods may offer a snapshot of gene expression patterns, they are extremely limiting. For example, the specific point in time chosen for isolating the cDNA from a tissue is limited by the development of the tissue itself. If an embryonic brain is not visible until the 7^{th} day, the earliest possible snapshot of embryonic brain development that can be obtained is on that 7^{th} day when the cells from that tissue are visible and can be isolated. This leaves a gap in time in which the gene expression patterns cannot be analyzed. Even when the tissue is visible, the task of isolating cDNA libraries from specific tissues at a myriad of developmental stages is daunting and requires a large amount of that specific tissue at various developmental stages.

Current cDNA library-generating strategies rely on the investigation of gene transcripts present within a given population of cells at the precise moment when RNA is extracted. While these methods are useful for studying static gene expression, they do not provide a temporal profile of dynamic gene expression that occurs during specific cellular processes.

Eisen et al. (1998) has described a cluster analysis for gene expression from DNA microarray hybridization using “ . . . standard statistical algorithms to arrange genes according to a similarity in pattern of gene expressions . . . ”. This method has been primarily used to group genes in clusters according to known similar function in the budding yeast, Saccharomyces cerevisiae, as well as in the human. This approach can be used for analyzing expression of novel genes; however, it requires knowledge of the function of the genes under study.

Another limiting aspect of current methods is that they do not use isolated systems. This creates a problem in determining which genes and gene expression patterns are part of which tissue type. For example, an embryonic brain contains both neural and non-neural tissue. Thus, it is impossible to determine whether a specific gene in a cDNA library isolated from an embryonic brain is associated with neurogenesis or is associated with the development of the non-neural tissue.

The method of single cell PCR has proved useful for studying gene expression in identified single brain cells (Van Gelder et al., 1990; Eberwine et al, 1992). This method relies on the production of amplified heterogeneous populations of RNA from limited quantities of cDNA. RNA from defined single cells is amplified following microinjection of primer, nucleotides and enzyme into single cells. Antisense RNA is amplified, and a second round of amplification generates more of the original material. The amplified RNA is used to generate cDNA libraries and/or probes. This method has been used for single cell molecular/genetic studies to generate expression libraries; however, amplification of RNA populations risks a significant amount of amplification of RNA fragments resulting from RNA degradation. Thus an incomplete gene profile may be incomplete and therefore not representative of the genes present at the stage of cell development being studied.

### 1.3 DEFICIENCIES IN THE PRIOR ART

None of the methods currently available offer an isolated system which can be used to determine gene expression patterns at infinite stages of development starting from a stem/precursor cell and proceeding through differentiated cells.

With the recent discovery of persistent neurogenesis in the adult mammalian (including human) brain, there is a need to identify new genes and factors that are involved in neural stem cell growth, differentiation, and the expression patterns of genes involved in such cellular growth cascades. Likewise there has not been a panel of cDNA pools available to use for determination of dynamic gene expression patterns in normal cellular processes; particularly in injury and disease as related to human genes specific for neural cells.

Thus, there is a need to develop micropanel arrays of cDNA pools that can be used to determine dynamic gene expression patterns in a variety of cellular processes and to identify new genes associated with developmental stages of cell maturation.

### 2.0 SUMMARY OF THE INVENTION

The present invention addresses several of the aforementioned deficiencies by providing novel temporally arrayed panels of neurosphere clones representing an isolated system derived from a single stem/progenitor cell. The stem/progenitor cells give rise to progeny cells to provide microclones from which cDNA can be isolated. cDNA isolation from these microclones can take place during any stage of microclone development Because the stage of microclone development and the type of microclone can be determined, cDNA libraries from different microclones can be compared. Gene expression patterns from microclones at different developmental stages can be compared and temporally ordered.

The invention in one aspect is concerned with methods of making and using cDNA libraries from microclones of proliferating stem and early progenitor cells. Because the cDNA can be isolated from these microclones at any stage of microclone development, the libraries generated allow gene expression patterns to be temporally ordered. Furthermore, a comparison of the genes expressed from cDNA libraries isolated from phenotypically different, or genotypically different microclones, or sets of microclones, can be used to identify new genes, and can be used to compare gene expression patterns among the different populations of microclones. In addition, when the microclones are derived from tumor cells, the disclosed methods can be used to discover and identify tumor specific genes and tumor specific gene expression patterns. This information will aid in the diagnosis, prognosis, and treatment strategy of a patient with the tumor from which the microclone is derived.

cDNA from microclones derived from neural and non-neural tissues can be compared to show differential gene expression between neural and non-neural tissues. cDNA libraries from microclones derived from injured brain cells can be compared with the cDNA libraries isolated from non-injured brain cells identifies genes involved in brain development, injury and regeneration.

In addition, cDNA libraries from stem/progenitor cells varying in genotype (e g., mutant, transgenic, wild-type) are useful in correlating gene expression among different types of cells. By analyzing changes in the pattern of gene expression in the presence and absence of drugs, drug effects on neural development may be determined.

Yet another aspect of the invention includes an algorithm method for temporal patterning of gene expression during neurogenesis. During cultivation of neurospheres under different conditions, neurosphere cells undergo differentiation through a variety of stages which mirror cell growth and differentiation seen in the developing brain in vivo. The creation of panels of cDNA libraries from different clones of neural stem/progenitor cells permits the temporal ordering of gene expression during neuron and glial growth and differentiation.

Another important aspect of the invention is the provision of an in vitro model for neurogenesis. An in vitro paradigm favors the generation and straightforward sampling of these “microsystems” for gene analysis and discovery. Such a model has conventionally been paralleled by producing huge tissue collections and cDNA libraries from literally a moment-to-moment sampling of embryonic brain tissue, in order to generate a model for gene discovery during neurogenesis achieved by the use of differentiating neurospheres in vitro. The present invention overcomes the need for such tedious and time-consuming tissue sampling by providing panels of microclones representing a continuous spectrum of early cell development and maturation from stem/progenitor cells.

In practicing the invention, microclones of cells to are exposed to agents that afford a rapid and reliable cDNA synthesis and amplification. Microclones are generated under particular defined culture conditions. These microclones contain multipotent cells representing different stages of neurogenesis which represent developmental gene expression as recapitulated in the microclones. Multipotent cells of the microclones develop and differentiate into different types of neurons and glia over time in vitro, and thus can be used as models for temporal variations in gene expression that profile the process of neurogenesis in vivo.

The method utilizes a culture technique that facilitates the isolation and characterization of stem, precursor, and progenitor cells from the central nervous system using suspension cultures, semi-solid media and anti-adhesive substrate, and factors that interfere with cell-cell and cell-substrate interactions (Kukekov et al., 1999). Using such culture approaches to produce different cell clones derived from single and distinct stem/progenitor cells, genetic material is readily isolated from these clones. Patterns of gene expression may be compared and, importantly, arranged temporally. The method provides a novel system for the discovery of new genes. The discovery of new genes is relevant to human and other mammalian genome analysis, as well as for the development and production of new factors and reagents that enhance neuropoiesis for purposes of stem cell biology and eventual cell replacement therapies for neurological diseases, traumatic injuries, neurodegenerative diseases and brain neoplasms.

Another aspect of the invention is the preparation of microarrays. Microarrays from cDNA fragments facilitate screening of numerous genes that define phenotype such as neurons and glial cells, as well as for developmental genes including homeobox, basic helix-loop-helix, transcription factor, apoptotic and anti-apoptotic genes. Such fragment analyses offer a precedent to yeast two-hybrid systems currently used for gene discovery and screening for associated gene/protein expressions.

Monoclonal panels comprising developmental stage profiles further comprise the invention. cDNA can be generated from single neurospheres. The diversity of neurospheres from single brain specimen dissociations provides a model system for a systematic arrangement of neural gene expression according to a temporal profiling approach. New genes may be discovered using these methods as a result of the cross-comparisons of temporally close (neighbors) neurospheres that are in different states of differentiation. The potential for large-scale analyses of large numbers of clones and libraries is now possible with the adaptation of these methodologies to 96-well micro-titer formats. The disclosed cDNA panels may be used for generating large numbers of microarrays for gene discovery. The format is amenable to high throughput (e.g., DNA chip, the use of automated or robotic assay systems and readers) analyses.

In particular aspects of the invention novel genes associated with different stages of cell maturation may be identified from brain tissue isolated from patients having a variety of neurological disorders, such as Parkinson disease, Alzheimer's disease, Huntingtons disease or brain tumors. The method can be used to identify stages and patterns of gene expression associated with the cause of the disease.

### 3.0 BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1 is a photomicrograph of microclones; the inset in the figure is a photomicrograph of a single adult brain microclone, scale bar=20 μm;

FIG. 2 is a flowchart for the method and application of cDNA microarray pools for neural gene discovery;

FIG. 3 is a representative sample of RT-PCR from single versus multiple microclones, a photomicrograph of a gel showing the presence of gene transcripts for various neuronal, glial, and developmental markers of individual versus multiple brain microclones; RT-PCR™ products are shown from individual compared to populations of neurospheres looking at a variety of precursor (e.g., nestin), extracellular matrix (e.g., tenascin), neuronal (e.g., MAP-2, neurofilament), glial (e.g., GFAP) gene markers. RT-PCR™ from adult neurospheres showing transcripts for different developmental and cell-specific markers. RT-PCR™ from single (A-F) and a population (G-J) of neurospheres. On either side of A-J are DNA ladders. A, β-actin; B, GFAP; C, tenascin (nested primer); D, nestin (nested primer); E, neurofilament-m; F, HuD (a neuron and neuron progenitor marker); G, β-actin; H, tenascin; I, GFAP (nested primer); and J, nestin (nested primer).

FIG. 4 is a diagram of the iterative algorithm used to perform the comparative analysis;

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### 4.0 DETAILED DESCRIPTION OF THE INVENTION

For the first time it is possible to create a detailed profile of neurogenesis represented by a temporal spectrum of developmental genes. Clones originating from brain stem/progenitor cells represent distinct stages of mammalian brain development known as neuromorphogenesis. Panels of cDNA libraries from multiple neurospheres at different stages of growth and differentiation contain transcripts of all genes which are involved only in neural cell division, expansion, growth, differentiation, and survival/death. The cloning process avoids isolation, growth and expansion of any non-neural cells such as vascular- or connective tissue-associated cells and identifies temporally regulated gene expression in vitro that recapitulates neuromorphogenetic gene expression in vivo. These panels allow identification of (a) novel genes involved in continual neuron proliferation (neurogenesis); and (b) temporal profiling of variance in gene expressions, including the switching-on and off of known genes, involved in the process of neurogenesis.

Individual neurosphere cDNA library production, along with application of a novel iterative algorithm characterizes and orders genes expression in single brain clones based on a temporal ordering sequence that is different from clustering based on similar gene function. Since neural gene discovery has relied on creation of libraries from whole tissue specimens at particular stages of maturity, and include a variety of cellular types (including non-neural, vascular-connective tissue-associated cells), the approach described here using neurospheres as Microsystems for gene analyses is a highly controlled in vitro paradigm for the acquisition of developmentally diverse cDNA libraries. This represents an entirely new approach for genetic analyses of what was once believed to be too complex a system (i.e. brain) to initiate gene discovery studies.

4.1 Neurogenesis

Recent work has established that existence of clonogenic stem/progenitor cells in the adult human brain capable to form clones (neurospheres) in vitro (Kukekov et al., 1999), supporting neurogenesis in the adult human brain in vivo (Eriksson et al., 1998). This study also traced the origin of multipotent stem/progenitor cells from two sources in the adult human brain —the SEZ and the hippocampus, and found common gene expression within neurospheres from both structures.

The pluripotency and self-renewal capabilities of neuropoietic cells has become a focus of attention despite a paucity of markers needed to categorize these cells in a manner similar to hematopoietic cells. It is believed that subsets of neurospheres express distinct markers, since stem cells from hematopoietic and other germinal sources can be immunolabeled with the different carbohydrate-recognizing stage-specific embryonic antigen (SSEA) antibodies (Thomson et al., 1998; Shamblott et al., 1998). Immunophenotypic analysis of cultured embryoid bodies reveals a “programmed sequence of cell surface marker display” associated with the development of embryonic cell lineages (Ling and Leben, 1997).

A similar pattern of distinct molecular expressions has been shown to accompany neurosphere growth and maturation in vitro. Other aspects of neurosphere architecture that provide insights into cell/molecular interactions within these structures are related to the appearance of new genes during development of neural cells. It has been demonstrated that each neurosphere represents a potentially distinct clonal unit that arose from a stem/progenitor cell in a particular stage of its maturation or evolution. Each neurosphere is thought to represent the clonal expansion of a cell that may have originated during a distinct ontological stage of neural development. Heterogeneous populations of neurospheres (Kukekov et al., 1997) could be composed of mixtures of cells in miscellaneous stages of differentiation.

4.2 Cell Markers

Markers of hematopoietic and neuropoietic cells are related to the molecular bases of stem/progenitor cell fate and growth. Comparative studies of neurospheres could be performed by screening for many of the same immunomarkers used, for example, in the studies of ES cells (e.g., SSEA-1, 3, 4; alkaline phosphatase, TRA-1-60, TRA-1-81) (Thomson et al., 1998; Shamblott et al., 1998). Another approach is to analyze a limited number of genes believed to be fate markers in other primitive cells and systems, including stem cells in Drosophila (Doe et al, 1998), and genes expressed during each ecodermal versus neural commitment (e.g., noggin, Xnr) (Chang and Hemmati-Brivanlou, 1998).

On the other hand, neurospheres themselves offer a starting-point from which to begin gene discovery studies, since markers and genes expressed by some of the most primitive hematopoietic (e.g., CD34, stem cell factor) and neuropoietic (certain cytoskeletal proteins, e.g., nestin (McKay, 1997), tenascin and Pax-6 (Kukekov et al., 1999) stem progenitor cells can be used to isolate these cells for subsequent gene and molecular analyses. For example, using the cell surface marker PSA-NCAM, Rao and collaborators (Mayer-Proschel et al., 1997) have used a panning method to isolate neuronal-restricted precursor cells, and using such approaches it is possible to profile genes involved in the commitment and maturation of particular populations of CNS cells.

4.3 In Vitro Neurogenesis Model

The inventors have used individual neurospheres in different states of in vitro maturation/differentiation as isolated Microsystems for identifying new genes. These neurospheres are obtained from adult mouse brain dissociations, as well as from human biopsy specimens (Kukekov et al., 1999), and because of their different states of development in culture, a continuum of maturing neurospheres may be viewed as a model of isolated germinal matrix zone that produces all CNS cell types in vivo. Thus panels of cDNA libraries from a spectrum of differentiating neurospheres contain a full set of transcripts of genes responsible for cell proliferation and fate decisions as seen during in vivo neuromorphogenesis. Moreover, because it is possible to generate neurospheres from autopsy specimens with surprisingly extended postmortem intervals (e.g., even up to 5 days) (Laywell et al., 1999). Panels of cDNA libraries can be created from neurologically rare abnormal stem/progenitor cells (e.g., neurodegenerative diseases such as Parkinson's, Alzheimer's and Huntington's disease as well as those derived from tumors).

4.4 Microclonal cDNA Pools

The technology of the inventive method described here is based on a procedure to generate pools of complementary deoxyribonucleic acids (cDNA) from mRNA of microclones where a “cDNA pool” can be defined as an uncloned cDNA library. In this system, a microclone is a culture-generated geometric structure wherein all the progeny are descendants of a single stem/progenitor cell (Reynolds and Weiss, 1992; Reynolds and Weiss, 1996; Kukekov et al, 1997). The cDNA libraries generated according to the invention make it possible to compare microclones where each microclone represents distinct stages of mammalian brain development. This method may be applied to non-neural tissues as well.

4.5 Microclones

Microclones are isolated systems that can be created from a variety of cell types in which all of the cells in the microclones are the progeny of a single primogenitor or ancestor cell. Microclones derived from neural stem/progenitor cells are clonal structures also referred to as neurospheres. During their growth under different conditions, neurosphere cells undergo differentiation through the stages which mirror cell growth and differentiation as seen during brain development in vivo. Anatomical and molecular ultrastructural analysis of these brain cell microclones reveal a diverse population of neural morphotypes undergoing significant changes during their in vitro cultivation, and these changes reflect distinct cellular and molecular interactions (Kukekov, et al., 1999). Brain cell microclones can therefore be viewed as isolated, miniature models of neurogenesis. Similarly, tumor cell microclones in which all of the cells in the microclone are the progeny of a single ancestor tumor cell can also be created.

4.6 Temporal Ordering of cDNA Libraries

The cDNA libraries from these microclones are arranged in temporal order based on the microclone from which the cDNA library was obtained. Microclonal cDNA pools can be generated and arranged according to the expression of any known gene. For example, cDNA pools can be arranged according to the expression of genes including, but not limited to, developmental or oncogenic genes. Thus, the generation of uncloned cDNA libraries constituting an ordered array of genes is amenable to perpetual analyses of transcripts present in a given microclone from any tissue at any stage of development or differentiation. For example, brain cell microclones representing early stages of neurogenesis yield cDNA libraries containing genes expressed during early neurogenesis. Brain cell microclones representing late stages of neurogenesis yield cDNA libraries containing genes expressed during late neurogenesis. By isolating the cDNA from brain cell microclones at these various stages, the genes within these libraries can be arranged according to when they are expressed at different stages of neuromorphogenesis.

4.7 Differential Gene Expression Panels

A panel of cDNA libraries derived from microclones at all stages of development, by definition, contains the transcripts of all genes involved in development including neural cell propagation, growth, differentiation, survival, and death. Therefore, such panels of cDNA libraries can be used in the discovery of new genes involved in the process of neurogenesis. They can also be used to analyze the chain of events that lead to the switching on and off of both novel and known genes involved in the process of neurogenesis. When cDNA isolated from microclones derived from normal brain cells is compared with cDNA isolated from microclones derived from abnormal brain cells, analysis of the differential gene expression patterns between these two microclone population identifies genes and pathways of activation involved in normal and abnormal brain development and brain function.

Comparisons of any two cDNA pools can be performed using various differential methods such as, but not limited to, representation different analysis (RDA), suppression subtractive hybridization (SSH), and enzymatic degrading subtraction (EDS). Thus, transcripts or fragments of transcripts can be discovered that are specific for one microclone versus another. This comparative or differential method affords the opportunity to sequence newly-discovered gene fragments for searching and comparing with known sequence data.

4.8 Ordering of Microarrays

An iterative gene screening process for diverse clones, in particular brain clones, is also encompassed within the present invention. After generating sequences, or fragments of sequences, primers are generated and all cDNA pools are screened for the presence of a particular gene, or gene fragment. This then allows a reorganization of the temporal pattern of gene expression by particular microclones. This process proceeds in an iterative fashion for n-1 times, when n is the number of microclones or pools included in a panel. This subjects microclones to repeated screenings to continually rearrange the extent of maturation or differentiation of any given microclone.

Following the ordering of genes in microarrays, the fragments for known and unknown genes can be rearranged in an order that more reliably reflects the precise timing of a particular cellular process. For example, the ordering of a particular set of brain cell microclones leads to the temporal gene expression pattern for neurogenesis. These genes or gene fragments can then be put into any number of commercial arrays such as DNA chips. A defined population of marker genes may be used as a primary method of sorting for subsequent gene discovery studies. The method and set of gene markers used is chosen based on the particular microclone population or populations selected. Subsets of arrays will exist where the cDNA pools reveal both neuronal and glial markers.

Following microarray analysis, specific genes or gene fragments can be used to generate oligonucleotide and riboprobes for in situ hybridization and in situ RT-PCR studies to confirm the presence of gene expression and identify the cellular sources of this expression within identified microclones. This may be used in conjunction with other methods including double-label immunocytochemistry.

Microarrays are useful for the classification of genes, in particular neural genes that can number in the thousands. This method is also applicable to studies of non-neural tissues as well, including developmental genes, oncogenic genes, embryonic stem cell genes, and primordial germ cell genes. Indeed, any cells which can be propagated as clones can be used.

4.9 Isolation and Characterization of Genes

Once the cDNA libraries are generated from microclones, the expressed genes are characterized. Following the generation of cDNAs, one can use expressed sequence tags (ESTs) (Polymeropoulos et al., 1992). Others have developed procedures to quickly assign chromosomal position of these ESTs using computer programs to establish chromosomal regions that are likely not to be interrupted by introns in genomic DNA. PCR and oligonucleotide primers may then used to amplify such regions by using DNA template from somatic cell hybrid chromosomal panels. Chromosomal assignment of cDNAs is then established following analysis of the segregation of amplified products in particular panels. Thousands of ESTs can be studied from developing human brain cDNA clones by focusing on the clones in an unbiased manner, then generating profiles of transcriptional activity of the brain at different developmental stages (Adams et al., 1993).

For quantitative expression measurements of corresponding genes, microarrays of cDNAs may be prepared with high-speed robotic printing on glass or nylon (Schena et al., 1995). Microarrays with sequences representative of most, or even all, human genes permit expression analysis of the entire human genome in a single reaction (Schena et al, 1998). Such information can be used to map genomic DNA clones as well as search for polymorphisms. Labeled probes are used to establish complementary binding and hence analyze large numbers of parallel gene expression. A sample of DNA is amplified by PCR, and a fluorescent label is inserted and hybridized to the microarray (Ramsay, 1998). Analysis of multiple DNA sequences can be accomplished using fiber-optic biosensor arrays (Ferguson et al., 1996), including the potential for quantitative analysis. Quantitative analysis can also be performed using calorimetric detections and computer-assisted image analysis (Chen et al., 1998). These DNA chip technologies previously have not been applied to neurospheres. The neurospheres can be used in conjunction with the cDNA obtained by the disclosed methods.

Neither method has been applied to clones of brain cells (neurospheres). Identification of different gene expression is a goal of both methods, and such differential or subtractive methods (e.g., representational difference analysis, RNA, or cDNAs) can be coupled with any microarray approach (Welford et al, 1998).

The cDNA microarray method of the present invention is distinct but compatible with differential display and subtractive methods for determining differences in gene expression across two different brain clones. Rather, the disclosed methods are employed to confirm distinct gene expressions across clones and isolate novel transcripts that can later be sequenced for confirmation of gene discovery.

There are technological caveats associated with the characterization of messages underlying development and maturation of developing neural cells by relying on subtractive hybridization of cDNA with mRNA or subtractive hybridization of cDNA libraries. This representational difference analysis (RDA) (Lisitsyn et al., 1993; Hubank and Schatz, 1994) is a method that overcomes many of the technical problems that rely on the RDA process where subtraction is coupled to amplification. Where differential display relies on the amplification of fragments from all represented mRNA species, RDA eliminates the fragments present in two different populations and leaves only the differences. It is an advantageous method for use with the cDNA microarrays. By establishing enriched libraries of differentially-expressed genes, the iterative process applied to brain cell microarrays affords a quick and reliable method for the concomitant screening of thousands of gene fragments. Other differential methods including enzymatic degrading subtraction (EDS) (Zeng et al., 1994) and suppression subtractive hybridization (SSH) (Diatchenko et al., 1996), may also be employed for differential gene expression studies of microarrays from brain neurospheres.

4.10 Mouse Neurospheres From Dissociated Brain

Using a protocol described in Kukekov et al. (1997), two types of neurospheres have been isolated from adult mouse neurospheres that give rise to both neurons and glia. Both light and electron microscopic studies have characterized the type of cells that reside within a novel (type I) and a well-characterized (type II) proliferative neurosphere. There appears to be a continuum of neurosphere development, with both early type I (nestin negative) and late type II neurospheres having cells with distinct morphologies and biochemistries.

Type I neurospheres appear spontaneously in suspension cultures. These neurospheres are phase-dark, spherical bodies that become larger and brighter with time. EM reveals that type I neurospheres consist of rings of small, tightly apposed cells that sometimes surround a core of flocculent material that may be debris from dying cells as well as extracellular matrix. The type I cell has many organelles including endoplasmic reticulum, Golgi apparatus, dense bodies, and mitochondria. The early forms of type I neurospheres are characterized by a sharp, continuous outer border. In contrast, late type I neurospheres often display a discontinuous outer border with cells beginning to protrude from the neurosphere. Type I neurospheres do not readily attach to either plastic or laminin-coated substrates.

Immediately after they appear in culture, type I neurospheres are immunonegative for cell-specific markers including astrocytic GFAP, the intermediate filament protein of putative neural stem cells, nestin, neuronal β-III tubulin, and L1; however, these neurospheres contain living cells as demonstrated by the positive labeling of nuclei with PI. After approximately two weeks in vitro, some cells of type I neurospheres become immunopositive for nestin, but remain immunonegative for GFAP and β-III tubulin. Over time, conversion of type I to type II neurospheres has been shown (Kukekov et al., 1997).

The early form of type II neurospheres resembles late type I neurospheres, except they contain larger cells, are phase-brighter, and have a more discontinuous border with more distinct cellular protrusions that become even more apparent in late type II neurospheres. EM of type II neurospheres shows cells that appear to be more differentiated than type I cells except that their cytoplasm is less electron dense than type I. Type II neurospheres readily attach to plastic and laminin substrates. After attaching, cells migrate out of the neurosphere and begin to elaborate processes. Type I and II neurospheres can also be generated from dissociations of the isolated adult SEZ in addition to whole brain, or from neonatal brain.

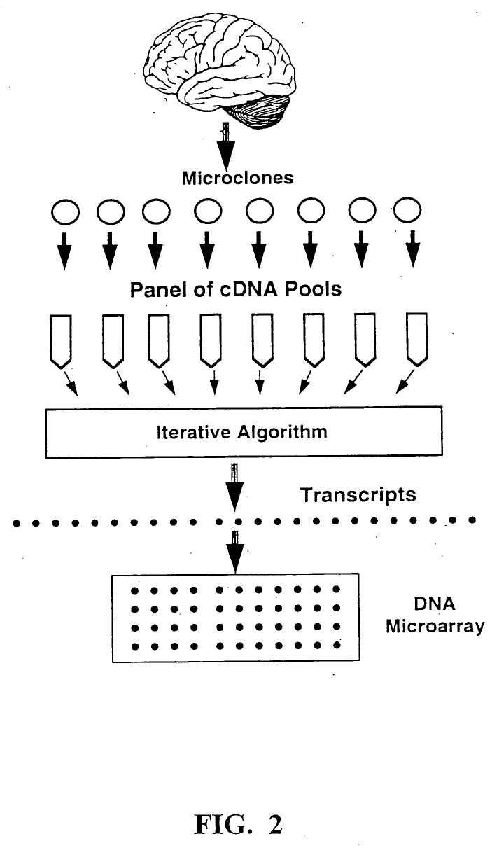

Type II neurospheres are immunopositive for nestin, GFAP, and β-III tubulin (FIG. 2E and FIG. 2F of Kukekov et al., 1997). When plated on plastic or laminin-coated substrates, type II neurospheres attach readily and produce a number of process-bearing cells that migrate away from the neurosphere to form a single layer of cells that are immunopositive for a variety of cell-specific markers including GFAP, nestin, L1, and β-III tubulin. Type I and II neurospheres, and neurons from these neurospheres, are proliferative (they take up BrdU). Finally, it has been shown that neurospheres can be generated from adult mouse brain tissue with even extended postmortem intervals (PMI), e.g., 140 hr (Laywell et al., 1999).

4.11 Human Neurospheres

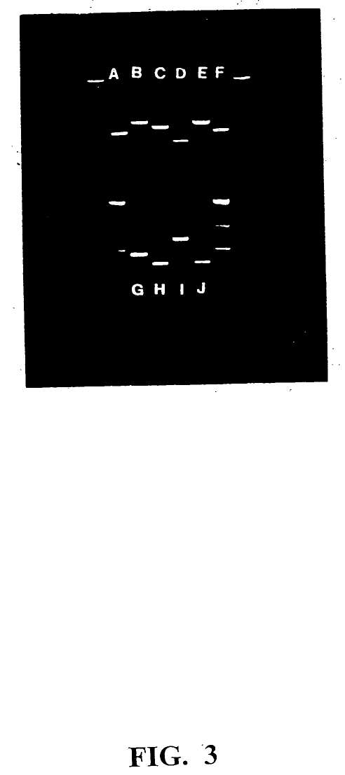

Type I and II neurospheres were generated from the adult human temporal lobe (biopsy specimens from temporal lobotomies performed for intractable epilepsy). Light microscopic immunocytochemistry and EM studies reveal neurosphere and cell types that have many characteristics in common with those seen from the adult mouse. Neurosphere cells also appear to be immunopositive for tenascin (see FIG. 3 of Kukekov et al., 1999), suggesting that in addition to the dense ECM expression by putative mature astrocytes in the SEZ rostral migratory pathway in vivo, that immature cells of neurospheres also express ECM proteins that can affect their growth and differentiation.

4.12 RT-PCR of Clonal Neurospheres

The presence of precursor, developmental, glial and neuronal phenotype markers has been associated with single and populations of early type I-late type II neurospheres. Kukekov et al. (1999) reported RT-PCR products from single and multiple neurospheres generated from mouse or human brain tissue as described (Suslov et al., 2000). Bands that correspond to appropriate base pair numbers indicate the presence of tenascin, nestin (related to the presence of an immature cell or perhaps the presence of the intermediate filament protein in reactive astrocytes (Lin et al., 1995)). GFAP, neuron specific enolase, and Pax transcription factor gene expression is observed in neurospheres.

This is the first evidence provided of a synthesis and expression of tenascin by SEZ stem/precursor cells although this has been shown in mature astrocytes in the SEZ migratory pathway (Thomas et al., 1996). The paired box genes, the Pax family, have been shown to be expressed in different parts of the developing brain and at different times in vivo (Stoykova and Gruss, 1994). This family is useful as a standard marker of the potential different states of maturity of individual clones.

RT-PCR on early type I-late type II neurospheres indicates that one is able to initially group these neurospheres based on phase microscopic morphology (e.g., phase dark versus phase bright), size, and time in culture to initially screen for the most immature versus mature neurosphere forms, and some of the same transcripts are expressed in one neurosphere as seen in several neurospheres of the same type. Some of the earliest neurospheres do not reveal transcripts for nestin, e.g., while some of the most “mature” neurospheres show heterogeneity for various neuronal markers. Some express tyrosine hydroxylase, GluR5 but not GluR6 and some express ChAT, supporting the usefulness of the method for distinguishing different precursor and neuronal/glial progenitor cells within neurospheres which exhibit different states of maturation or alternatively neurospheres that have arisen from different types of stem/precursor cells. Differences in the expression of these genes in stem/progenitors or clones from different neurogenic zones demonstrates the use of this method to further characterize additional genes expressed by one population of clones versus another.

4.13 Nucleic Acid Segments

The present invention also provides for isolated nucleic acid molecules comprising nucleotide sequences encoding the amino acid sequences of genes isolated and identified by the disclosed methods. Fragments and variants of the disclosed nucleotide sequences and proteins encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein. Fragments of a nucleotide sequence may range from at least about 20 nucleotides, about 50 nucleotides, about 100 nucleotides, and up to the entire nucleotide sequence.

A fragment of a nucleotide sequence that encodes a biologically active portion of a protein will encode at least 15, 25, 30, 50, 100, 150, 200, 250, 300, 350, 400, 450, 500, 600, 700, 800, or 1000 contiguous amino acids, or up to the total number of amino acids present in a full-length protein of the invention. Fragments of the nucleotide sequence useful as hybridization probes for PCR™ primers generally need not encode a biologically active portion of a protein.

A fragment of a nucleotide sequence may encode a biologically active portion of a protein, or it may be a fragment that can be used as a hybridization probe or PCR™ primer using methods disclosed below. A biologically active portion of a protein can be prepared by isolating a portion of one of the nucleotide sequences of the invention, expressing the encoded portion of the protein (e.g., by recombinant expression in vitro), and assessing the activity of the encoded portion of the protein. Nucleic acid molecules that are fragments of a nucleotide sequence comprise at least about 16, about 17, about 18, about 19, about 20, about 21, about 22, about 23, about 24, about 25, about 26, about 27, about 28, about 29, and about 30 or so contiguous nucleotides. Slightly longer sequences include those that comprise at least about 31, about 32, about 33, about 34, about 35, about 36, about 37, about 38, about 39, about 40, about 41, about 42, about 43, about 44, about 45, about 46, about 47, about 48, about 49, or about 50 or so contiguous nucleotides. Still longer sequences include those that comprise at least about 51, about 52, about 53, about 54, about 55, about 56, about 57, about 58, about 59, about 60, about 61, about 62, about 63, about 64, about 65, about 6, about 67, about 68, about 69, or about 70 or so contiguous nucleotides. When it is desirable to identify even longer segments that comprise still longer contiguous nucleic acid sequences from or so contiguous nucleotides, one may prepare polynucleotides that comprise about 75, about 80, about 85, about 90, about 95, about 100, about 125, about 150, about 175, about 200, about 225, about 250, about 275, about 300, about 325, about 350, about 375, about 400, about 425, about 450, about 500, about 550, about 600, about 650, about 700, about 750, about 800, about 850, or so nucleotides, and even those comprising up to and including the number of nucleotides present in a nucleotide sequence.

By “variants” are intended substantially similar sequences. For nucleotide sequences, conservative variants include those sequences that, because of the degeneracy of the genetic code, encode a designated amino acid sequence of a protein. Generally, nucleotide sequence variants of the invention will have at least 40%, 50%, 60%, 70%, generally, 80%, preferably 90%, 95%, 98% sequence identity to its respective native nucleotide sequence.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

These nucleotide sequences can be used to isolate other homologous sequences. Methods are readily available in the art for the hybridization of nucleic acid sequences. To obtain other sequences, the entire polypeptide sequence or portions thereof may be used as probes capable of specifically hybridizing to corresponding coding sequences and messenger RNAs. To achieve specific hybridization under a variety of conditions, such probes include sequences that are unique and are preferably at least about 10 nucleotides in length, and most preferably at least about 20 nucleotides in length. Such probes may be used to amplify the protein coding sequences of interest by the well-known process of polymerase chain reaction (PCR™). This technique may be used to isolate additional coding sequences or as a diagnostic assay to determine the presence of coding sequences.

Such techniques include hybridization screening of plated DNA libraries (either plaques or colonies) (Sambrook et al., 1989) and amplification by PCR™ using oligonucleotide primers corresponding to sequence domains conserved among the amino acid sequences (Innis et al., 1990).

Hybridization of such sequences may be carried out under conditions of reduced stringency, medium stringency or even stringent conditions (e.g., conditions represented by a wash stringency of 35-40% Formamide with 5× Denhardt's solution, 0.5% SDS and 1×SSPE at 37° C.; conditions represented by a wash stringency of 40-45% Formamide with 5× Denhardt's solution, 0.5% SDS, and 1×SSPE at 42° C.; and conditions represented by a wash stringency of 50% Formamide with 5× Denhardt's solution, 0.5% SDS and 1×SSPE at 42° C., respectively), to DNA encoding the proteins disclosed herein in a standard hybridization assay (Sambrook et al., 1989). In general, polynucleotide sequences which encode a polypeptide as disclosed herein and which hybridize to one or more of the polynucleotide sequences disclosed herein will be at least 50% homologous, 70% homologous, and even 85% homologous or more with the disclosed sequence. That is, the sequence similarity of sequences may range, sharing at least about 50%, about 70%, and even about 85% sequence similarity.

Methods of alignment of sequences for comparison are well known in the art. Optimal alignment of sequences for comparison may be conducted by the local homology algorithm of Smith et al., (1981); by the homology alignment algorithm of Needleman et al., (1970); by the search for similarity method of Pearson et al., (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Drive, Madison, Wis., USA; the CLUSTAL program is well described by Higgins et al., (1988); Higgins et al., (1989); Corpet et al., (1988); Huang et al., (1992), and Person et al., (1994); preferred computer alignment methods also include the BLASTP, BLASTN, and BLASTX algorithms (Altschul et al., 1990). Alignment is also often performed by inspection and manual alignment.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. to about 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, stringent conditions of hybridizations are exemplified by wash conditions in which the salt concentration is about 0.02 M at pH 7 and the temperature is at least about 50° C., about 55° C., or even about 60° C. or so. However, nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman et al., (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

Proteins may be altered in various ways including amino acid substitutions, deletions, truncations, and insertion. Methods for such manipulations are generally known in the art. For example, amino acid sequence variants of proteins can be prepared by mutations in the DNA. Methods for mutagenesis and nucleotide sequence alterations are well known in the art (Kunkel, 1985; Kunkel et al., 1987; U.S. Pat. No. 4,873,192; Walker and Gaastra, 1983).

It is intended that the genes and nucleotide sequences of the invention include both the naturally occurring sequences as well as mutant forms. Likewise, the proteins of the invention encompass both naturally occurring proteins as well as variations and modified forms thereof. Such variants will continue to possess the activity. Obviously, the mutations that will be made in the DNA encoding the variant must not place the sequence out of reading frame and preferably will not create complementary regions that could produce secondary mRNA structure (see e.g., European Patent Application Publication No. 75,444, specifically incorporated herein by reference in its entirety).

4.14 Expression Vectors

Expression vectors comprising at least one polynucleotide operably linked to an inducible promoter may be readily constructed from nucleic acid sequences isolated by the disclosed methods. Thus, in one embodiment an expression vector is an isolated and purified DNA molecule linked to a promoter that expresses the gene, which coding region is operatively linked to a transcription-terminating region, whereby the promoter drives the transcription of the coding region.

As used herein, the term “operatively linked” means that a promoter is connected to a nucleic acid region encoding functional RNA in such a way that the transcription of that functional RNA is controlled and regulated by that promoter. Means for operatively linking a promoter to a nucleic acid region encoding functional RNA are well known in the art.

The choice of which expression vector and ultimately to which promoter a polypeptide coding region is operatively linked depend directly on the functional properties desired, e.g., the location and timing of protein expression, and the host cell to be transformed. These are well known limitations inherent in the art of constructing recombinant DNA molecules. However, a vector useful in practicing the present invention is capable of directing the expression of the functional RNA to which it is operatively linked.

RNA polymerase transcribes a coding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA).

A variety of methods have been developed to operatively link DNA to vectors via complementary cohesive termini or blunt ends. For instance, complementary homopolymer tracts can be added to the DNA segment to be inserted and to the vector DNA. The vector and DNA segment are then joined by hydrogen bonding between the complementary homopolymeric tails to form recombinant DNA molecules.

4.15 DNA Segments as Hybridization Probes and Primers

In another aspect, DNA sequence information provided by the invention allows for the preparation of relatively short DNA (or RNA) sequences having the ability to specifically hybridize to gene sequences of the selected polynucleotides disclosed herein. The probes may be used in a variety of assays for detecting the presence of complementary sequences in a given sample, and in the identification of new species or genera of encoding genes.

In certain embodiments, it is advantageous to use oligonucleotide primers. The sequence of such primers is designed using a polynucleotide of the present invention for use in detecting, amplifying or mutating a defined segment of the disclosed nucleic acid segments from a sample using PCR™ technology. To provide certain of the advantages in accordance with the present invention, a preferred nucleic acid sequence employed for hybridization studies or assays includes sequences that are complementary to at least about 31 to 50 or so long nucleotide stretch of. A size of at least 31 nucleotides in length helps to ensure that the fragment will be of sufficient length to form a duplex molecule that is both stable and selective. Molecules having complementary sequences over stretches greater than 31 bases in length are generally preferred, though, in order to increase stability and selectivity of the hybrid, and thereby improve the quality and degree of specific hybrid molecules obtained. One will generally prefer to design nucleic acid molecules having gene-complementary stretches of about 31 to about 40 or 50 or so nucleotides, or even longer where desired. Such fragments may be readily prepared by, for example, directly synthesizing the fragment by chemical means, by application of nucleic acid reproduction technology, such as the PCR™ technology of U.S. Pat. No. 4,683,195, and U.S. Pat. No. 4,683,202, (each specifically incorporated herein by reference in its entirety), or by excising selected DNA fragments from recombinant plasmids containing appropriate inserts and suitable restriction sites.

Where one desires to prepare mutants employing a mutant primer strand hybridized to an underlying template or where one seeks to isolate related gene sequences, functional equivalents, or the like, less stringent hybridization conditions will typically be needed in order to allow formation of the heteroduplex. In these circumstances, one may desire to employ conditions such as about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

In addition to the use in directing the expression of functional RNA of the present invention, the nucleic acid sequences contemplated herein also have a variety of other uses. For example, they also have utility as probes or primers in nucleic acid hybridization embodiments. As such, it is contemplated that nucleic acid segments that comprise a sequence region that consists of at least a 21 nucleotide long contiguous sequence that has the same sequence as, or is complementary to, a 21 nucleotide long contiguous DNA segment will find particular utility. Longer contiguous identical or complementary sequences, e.g., those of about 20, 21, 22, 23, 24, etc., 30, 31, 32, 33, 34, etc., 40, 41, 42, 43, 44, etc., 50, 51, 52, 53, 54, etc., 100, 200, 500, etc. (including all intermediate lengths and up to and including full-length sequences will also be of use in certain embodiments.

Other uses are also envisioned, including the use of the sequence information for the preparation of mutant species primers, synthetic gene sequences, gene fusions, and/or primers.

The use of a hybridization probe of about 14 or so nucleotides in length allows the formation of a duplex molecule that is both stable and selective. Molecules having contiguous complementary sequences over stretches of about 15, 16, 17, 18, 19, or 20 or more bases in length are generally preferred, though, in order to increase stability and selectivity of the hybrid, and thereby improve the quality and degree of specific hybrid molecules obtained. One will generally prefer to design nucleic acid molecules having gene-complementary stretches of about 21, 22, 23, 24, 25, 26, 27, 28, 29, or 30 or more contiguous nucleotides in length where desired.

Fragments may also be obtained by other techniques such as, e.g., by mechanical shearing or by restriction enzyme digestion. Small nucleic acid segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,195 and U.S. Pat. No. 4,683,202 (each incorporated herein by reference), by introducing selected sequences into recombinant vectors for recombinant production, and by other recombinant DNA techniques generally known to those of skill in the art of molecular biology.

Accordingly, the nucleotide sequences of the invention may be used for their ability to selectively form duplex molecules with complementary stretches of DNA fragments. Depending on the application envisioned, one may desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by about 0.02 M to about 0.15 M NaCl at temperatures of about 50° C. to about 70° C. Such selective conditions tolerate little, if any, mismatch between the probe and the template or target strand, and would be particularly suitable for isolating particular DNA segments. Detection of DNA segments via hybridization is well known to those of skill in the art, and the teachings of U.S. Pat. No. 4,965,188 and U.S. Pat. No. 5,176,995 (each incorporated herein by reference) are exemplary of the methods of hybridization analyses. Teachings such as those found in the texts of Maloy et al., 1994; Segal 1976; Prokop and Bajpai, 1991; and Kuby, 1994, are particularly relevant.

In general, it is envisioned that the hybridization probes described herein will be useful both as reagents in solution hybridization as well as in embodiments employing a solid phase. In embodiments involving a solid phase, the test DNA (or RNA) is adsorbed or otherwise affixed to a selected matrix or surface. This fixed, single-stranded nucleic acid is then subjected to specific hybridization with selected probes under desired conditions. The selected conditions will depend on the particular circumstances based on the particular criteria required (depending, for example, on the G+C content, type of target nucleic acid, source of nucleic acid, size of hybridization probe, etc.). Following washing of the hybridized surface so as to remove nonspecifically bound probe molecules, specific hybridization is detected, or even quantitated, by means of the label.

4.16 Biological Functional Equivalents

Modification and changes may be made in the structure of the protein-specific genes, promoters, genetic constructs, plasmids, and/or polypeptides of the present invention and still obtain functional molecules that possess the desirable biologically-active characteristics. The following is a discussion based upon changing the amino acids of a protein to create an equivalent, or even an improved, second-generation molecule. The amino acid changes may be achieved by changing the codons of the DNA sequence, according to the codons given in Table 1.

TABLE 1

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic Acid | Asp | D | GAC | GAU | | | | |
| Glutamic Acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUG | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences that encode said peptides without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those that are within ±1 are particularly preferred, and those within ±0.5 are even more particularly preferred.

It is also understood in the art that the substitution of like amino acids can be made effectively on the basis of hydrophilicity. U.S. Pat. No. 4,554,101, specifically incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions, which take several of the foregoing characteristics into consideration, are well known to those of skill in the art and include: arginine and lysine; glutamate and aspartate; serine and threonine; glutamine and asparagine; and valine, leucine and isoleucine.

4.17 Antisense Oligonucleotides Targeted to mRNA

Antisense compositions may be employed to negatively regulate the expression of a protein-encoding gene sequence in a host cell. The end result of the flow of genetic information is the synthesis of protein. DNA is transcribed by polymerases into messenger RNA and translated on the ribosome to yield a folded, functional protein. Thus, even from this simplistic description of an extremely complex set of reactions, it is obvious that there are several steps along the route where protein synthesis can be inhibited. The native DNA segment encoding a protein, as all such mammalian DNA strands, has two strands: a sense strand and an antisense strand held together by hydrogen bonding. The messenger RNA encoding a protein has the same nucleotide sequence as the sense DNA strand except that the DNA thymidine is replaced by uridine. Thus, antisense nucleotide sequences will bind to the mRNA encoding its polypeptide and inhibit production of the protein.

The targeting of antisense oligonucleotides to bind mRNA is one mechanism to shut down protein synthesis. For example, the synthesis of polygalactauronase and the muscarine type-2 acetylcholine receptor are inhibited by antisense oligonucleotides directed to their respective mRNA sequences (U.S. Pat. No. 5,739,119 and U.S. Pat. No. 5,759,829, U.S. Pat. No. 5,801,154; U.S. Pat. No. 5,789,573; U.S. Pat. No. 5,718,709 and U.S. Pat. No. 5,610,288, each specifically incorporated herein by reference in its entirety).

In illustrative embodiments, antisense oligonucleotides may be prepared which are complementary nucleic acid sequences that can recognize and bind to target genes or the transcribed mRNA, resulting in the arrest and/or inhibition of deoxyribonucleic acid (DNA) transcription or translation of the messenger ribonucleic acid (mRNA). These oligonucleotides can be expressed within a host cell that normally expresses a specific mRNA to reduce or inhibit the expression of this mRNA. Thus, the oligonucleotides may be useful for reducing the level of polypeptide in a suitably transformed host cell.

The oligonucleotides may comprise deoxyribonucleic acid, ribonucleic acid, or peptide-nucleic acid. In particular embodiments, the oligonucleotide comprises a sequence of at least nine, at least ten, at least eleven, at least twelve, at least thirteen, or at least fourteen, up to and including the full-length contiguous sequences. When longer antisense molecules are required, one may employ an oligonucleotide that comprises a sequence of at least fifteen, at least sixteen, at least seventeen, at least eighteen, at least nineteen, or at least twenty, up to and including the full-length contiguous sequences. Such antisense molecules may comprise even longer contiguous nucleotide sequences, such as those comprising about 21, about 22, about 23, about 24, about 25, about 26, about 27, about 28, about 29, or about 30 or so contiguous nucleotides.

4.18 Definitions

In accordance with the present invention, nucleic acid sequences include and are not limited to DNA (including and not limited to genomic or extragenomic DNA), genes, RNA (including and not limited to mRNA and tRNA), nucleosides, and suitable nucleic acid segments either obtained from native sources, chemically synthesized, modified, or otherwise prepared by the hand of man. The following words and phrases have the meanings set forth below.

A, an: In accordance with long standing patent law convention, the words “a” and “an” when used in this application, including the claims, denotes “one or more”.

Expression: The combination of intracellular processes, including transcription and translation undergone by a coding DNA molecule such as a structural gene to produce a polypeptide.

Promoter: A recognition site on a DNA sequence or group of DNA sequences that provide an expression control element for a structural gene and to which RNA polymerase specifically binds and initiates RNA synthesis (transcription) of that gene.

Structural gene: A gene that is expressed to produce a polypeptide.

Transformation: A process of introducing an exogenous DNA sequence (e.g., a vector, a recombinant DNA molecule) into a cell in which that exogenous DNA is incorporated into a chromosome or is capable of autonomous replication.

Transformed cell: A cell whose DNA has been altered by the introduction of an exogenous DNA molecule into that cell.

Vector: A DNA molecule capable of replication in a host cell and/or to which another DNA segment can be operatively linked so as to bring about replication of the attached segment. A plasmid is an exemplary vector.

Illustrative embodiments of the invention are described below. In the interest of clarity, not all features of an actual implementation are described in this specification. It will of course be appreciated that in the development of any such actual embodiment, numerous implementation-specific decisions must be made to achieve particular goals. Moreover, it will be appreciated that such a development effort might be complex and time-consuming, but would nevertheless be a routine undertaking for those of ordinary skill in the art having the benefit of this disclosure.

### 5.0 EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Methods have been developed for the isolation and in vitro expansion of diverse stem/progenitor cell populations from the adult mouse and human brain; and creation of cDNA libraries from individual clones or neurospheres. Using culture approaches for isolating and cultivating a wide range of other (e.g., hematopoietic) stem/progenitor cells that produce different colony-like structures similar to neurospheres (Scheffler et al., 1999), it is now possible to consistently generate large numbers of neurospheres from single tissue dissociations. These neurospheres are morphologically and biochemically different from each other thus representing a spectrum or range of differentiation states as well as originating from distinct stem/progenitor cells.

### 5.1.0 Example 1

### Preparation of Microclones

5.1.1 Type I Clones

Adult ICR or transgenic or mutant mice, or biopsy specimens from human temporal lobe (for epilepsy surgery), or brain specimens with significant (i.e. at least one day) postmortem intervals were used as tissue sources for dissociations. The brains were dissociated and cultured as follows. Extracted brain tissues were minced with a razor blade and washed in a mixture of ice-cold DMEM (Dulbecco's modified Eagle's medium, commercially available from a variety of vendors) and an antibiotic-antimycotic product (Sigma Chemical Co. Catalogue #A5955 (IOOX, St. Louis, Mo.; Gibco Brl, Grand Island, N.Y.). Brain pieces were transferred to a beaker containing 0.25% trypsin and EDTA (ethylenediaminetetraacetic acid) and mixed on a magnetic stir-plate for 15 minutes, triturated with a plastic pipette, filtered through sterile gauze, and collected in a 15 ml tube and centrifuged for 5 minutes at 1200 rpm. Cells were resuspended in DMEM/FI2 medium+NI supplement (a standard tissue culture medium available from a variety of vendors) plus 5% FBS (fetal bovine serum) and grown in suspension cultures by plating at high density on a non-adhesive substrate (tissue culture plastic coated with poly 2-hydroxyethyl methacrylate; Sigma Chemicals). Cells were fed every 3-4 days by centrifugation and resuspension in fresh medium.

The basic media for culturing type I cells comprises the following ingredients: Insulin (5 μg/mL), putrescine (100 μM), progesterone (20 μm, sodium selenite (30 μM), pituitary extract (20 μg/mL), transferring (100 μg/mL), and 5% fetal calf serum (FCS) in DNEM/FI2 media.

Type I cells appear only in suspension cultures containing a non-adhesive substrate such as poly 2-hydroxyethyl methacrylate. Some type II cells are also present in these cultures.

5.1.2 Type II Clones

Type II clones, similar to type I clones, were obtained from adult ICR, transgenic or mutant mice, or biopsy specimens from human temporal lobe (for epilepsy surgery). In addition, type II clones were also generated from the adult human brain, and from dead animals with long post mortem intervals when the animals were kept at 4° C.

The brains were dissociated and cultured as previously described for the generation of type I clones. Briefly, extracted brain tissues were minced, washed, and transferred to a beaker containing 0.25% trypsin and EDTA. After being mixed on a magnetic stir-plate for 15 minutes, the culture was triturated with a plastic pipette, filtered through sterile gauze, centrifuged, and resuspended in DMEM/F12 medium+NI supplement, plus 5% FBS, plus 20 μg/mL pituitary extract (from Gibco) and grown in suspension cultures by plating at high density on a non-adhesive substrate.

Cells were plated and fed as described above for the type I cells. However, the basic media described above (comprising insulin (5 μg/mL), putrescine (100 μM) progesterone (20 μM, sodium selenite (30 μM, pituitary extract (20 μg/mL), transferring (100 μg/mL), and 5% fetal calf serum (FCS) in DMEM/F12 media) also contained 10 ng/ML basic fibroblast growth factor (bFGF), and 10 ng/mL epidermal growth factor (EGF). Importantly, the culture media additionally contained 100 μM mercaptoethanol as a contact-limiting factor that reduces disulfide bonds (Herington, 1986). Cultures contained dense debris for 10-14 days. Mercaptoethanol was then removed from the medium after 10-14 days. Clones of type II were present in these cultures. Some type III clones were also present.

5.1.3 Type III Clones

Similar to the type I and type II clones, type III clones were obtained from adult ICR, transgenic or mutant mice, or biopsy specimens from human temporal lobe, or from the adult human brain or from dead animals with long post mortem intervals when the animals were kept at 4° C. The brain source was dissociated and the cells grown in the suspension culture described above either without contact inhibiting factors, or, with a contact inhibiting factor such as mercaptoethanol. The basic media for culturing type III cells was the same as that used for culturing type II cells; that is, the media comprised insulin (5 μg/mL), putrescine (100 μM), progesterone (20 EM), sodium selenite (30 μM), pituitary extract (20 μg/mL), transferring (100 μg/mL), 10 ng/mL basic fibroblast growth factor (bFGF), 10 ng/mL epidermal growth factor (EGF), and 5% fetal calf serum (FCS) in DMEM/F12 media Cells were fed every 3-4 days by centrifugation and resuspension in fresh medium. After removal of the contact limiting factor, both type II and Type III clones were apparent after 5-7 days. The type II clones eventually evolved into type III clones upon continued culturing in the absence of contact limiting factors.

Simply removing the contact inhibiting factors encourages differentiation by encouraging cell-cell contact However, differentiation of type III clones into neurons or glia is also encouraged by other additional factors, including the growth factors like β-fibroblast growth factor, epidermal growth factor, or factors that are contained within pituitary extract present in the basic type III culture media. Other growth factors such as brain-derived neurotrophic factor (BDNF), glial derived neurotrophic factor (GDNF), NT3, and ciliary neurotrophic factor (CNTF) may also encourage differentiation of the stem/precursor cells.

The following table summarizes the various methods to obtain the different stem/precursor cell types:

TABLE 2

| | Type I | Type II | | |
|---|---|---|---|---|
| Steps | clones | clones | Type III Clones | |
| Brain dissociation | + | + | + | + |
| Grow in suspension culture | + | + | + | + |
| Add contact inhibiting factor | + | + | + | + |
| (for ≦2 weeks) | | | | |
| Remove contact inhibiting factor | | + | + | |
| Culture or plate on plastic/laminin | | | + | + |
| coated substrate | | | | |

Phase microscopic examinations were used to initially categorize the cultured neurospheres into four categories that relate to a gross indication of maturity (differentiation); early type I (small, phase-dark spheres), late type I (large, phase-dark to phase-bright spheres), early type II (medium-sized, phase-bright spheres), to late type II or type III (large, phase-bright spheres). Representative neurospheres were cultivated for standard immunofluorescence analyses for confirmation of the presence of stem/progenitors, immature neurons and glia for use in single and double labeling protocols using conventional and confocal microscopic analysis (Kukekov et al., 1997; Kukekov et al., 1999). Antibodies for immunodetection included: the intermediate filament protein, nestin, present in stem/progenitor and immature neural cells, and reactive astrocytes (McKay, 1997); monoclonal antibody (Developmental Hybridoma Bank, Iowa City, Iowa); vimentin, an immature astrocyte intermediate filament protein (Developmental Hybridoma Bank); the astrocyte intermediate protein, glial fibrillary acidic protein (GFAP); A2B5 which recognizes oligodendrocyte precursor cells, e.g., 02A progenitors; the 04 antibody that recognizes immature oligodendrocytes (Developmental Hybridoma Bank); an antibody to the L1 adhesion molecule on young and mature neutrons (see FIG. 3H of Kukekov et al., 1999); and the β-III tubulin isoform of neurons (monoclonal antibody, Sigma, St. Louis, Mo.).

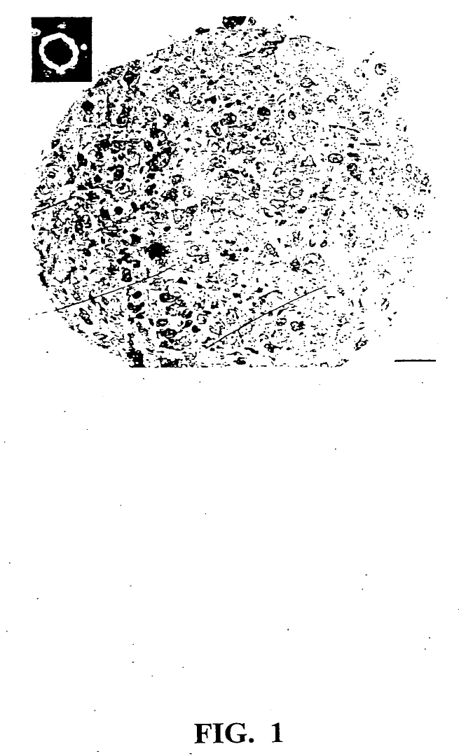

FIG. 1 shows the light and electron microscopic photographs of individual adult human brain microclones used as a source for cDNA pools and microarray generation. The inset in the figure is a photomicrograph of a single adult brain microclone using phase optics through an inverted light microscope. The electron micrograph figure shows numerous cell types tightly packed within an orbicular structure. There are up to several thousand cells that inhabit such a microclone, and these cells exhibit different morphologies indicative of multipotency of the microclone.

### 5.2.0 Example 2

### Preparation of cDNA Libraries

To maximize the yield and quality of RNA preparations, an appropriate method for the specific starting material was developed. Small quantities of mRNA can be measured by combining the sensitivity of PCR with specific generation of cDNA using reverse transcription. However, the time needed for extraction of intact RNA frequently surpasses the time involved in the RT-PCR procedure itself and may result in some loss of RNA. There have been protocols established that utilize RT-PCR without RNA isolation (Klebe et al., 1996).

Each brain cell microclone contains stem, precursor, and progenitor cells which give rise to neurons, astrocytes and oligodendrocytes. These cells are embedded in an extremely dense extracellular matrix (Kukekov et al., 1997) which is difficult to disrupt using conventional methods without losing material. There have been a number of protocols reported that utilize RT-PCR without RNA isolation, and advantages as well as drawbacks associated with these approaches have been discussed (Suslov et al., 2000). On the other hand, there have been several methods described for the release of RNA from different sources, using sonication, but all of these have included an additional step for RNA isolation. In an improved protocol, advantages of both approaches have been combined. Since the generation of neurospheres from valuable human brain specimens can be difficult, time-consuming, and does not always yield large numbers of these clones, modifications described in the following example were developed to provide a fast, reliable and sensitive method for isolating and detecting mRNA from single neurospheres without RNA extraction.

5.2.1 cDNA Pools From Microclones

An RT-PCR assay was streamlined by eliminating time-consuming procedures involved in RNA isolation. Manipulations were carried out in one tube and amplified cDNA was produced from the RNA available from small numbers of cells. Detection of mRNA transcripts for various genes in neurospheres, and even single cells within neurospheres was made possible using extremely small quantities of sonicate.

Each neurosphere contains at least 50400 stem/progenitor cells that can give rise to newly generated neurons, astrocytes and oligodendrocytes, and these cells are embedded in an extremely dense extracellular matrix (Kukekov et al., 1997; Kukekov et al., 1999) which make neurospheres difficult to disrupt by conventional methods, without losing material.

Single neurospheres, identified under the phase microscope as multicellular spherical structures, were each collected in a volume of 0.5 μl using a micropipette with filter tip. A single neurosphere was then transferred to 10 μl RNase-free water containing 5 μl RNase Inhibitor (Gibco BRL/Life Technologies, Gaithersburg, Md.). Neurospheres were then sonicated, using a Microtip Sonicator (Kontes, Vineland, N.J.), by gently touching the liquid surface for 5 sec., power 4, tune 2; the tubes were kept on ice before and after sonication. Temperature was measured in the test tubes, during sonication, using a digital minithermometer HH81 (Omega Engineering Inc., Stamford, Conn.). The optimal time range for sonication was determined to be 4-10 sec., since temperature increased during 10 sec. up to 55° C. It is not recommended to use less than 4 sec. to assure that RNA is completely released, but no more than 10 sec. since longer times decrease RNase inhibitor activity. When working with a number of samples, the sonicator microtip should be rinsed in a series of solutions (1 M HCl, 1 M NaOH, 1 M Tris-HCl, pH 7.5, double-distilled H₂O on ice) in order to avoid cross-contaminations as well as to cool the microtip. Neurospheres were transferred into a 0.5 ml tube, and the tube is snap-frozen in liquid nitrogen and then thawed in a 37° C. water bath. This procedure was repeated three times.

5.2.3 First Strand Synthesis of cDNA

SMART cDNA synthesis technology (CLONTECH) was used for the first-strand synthesis of cDNA with some modifications. A modified oligo(dT) primer (SDS) was used to prime the first-strand synthesis reaction, using the general procedure described by Clonetech. The resulting full-length, single stranded cDNA contained the complete 5′ end of the mRNA, as well as sequences that were complementary to the SMART oligonucleotide. The SMART anchor sequence and the poly A sequence served as universal priming sites for end-to-end cDNA amplification with SMART PCR™ primer. Because the SMART anchor sequence was necessary for PCR™, prematurely terminated cDNAs arising from incomplete RT activity, contaminating genomic DNA, and cDNA transcribed from poly A RNA were not exponentially amplified.

For each reaction 2 μl of 10 μm SDS primer and 2 ll of 10 μm SDS oligonucleotide was added to 10 μl of single-neurosphere sonicate. The tube was incubated at 72° C. for 2 min. and then chilled on ice. The product was then split to two tubes in aliquots of 7 μl. The first strand buffer (Gibco BRL) (25 mM Tris-HCl, pH 8.3; 37.5 mM KCl; 1.5 mM MgCl₂), 1 mM dNTP (CLONTECH), 3 mM MgCl₂ (Sigma), 5 U RNase Inhibitor (Gibco BRL) was added to each tube (all concentrations are final). The total volume of reaction was 2 μl in each tube. Both tubes were incubated at 42° C. for 5 min., then 1 μl of Superscript (Gibco BRL) was added and tubes incubated at 42° C. additionally for 1 hr.

The volumes from both tubes were transferred to a new tube when 100 μl of 1×TE was added, and were incubated at 70° C. for 15 min.

The first-strand cDNA pool was used for LD (Long Distance) amplification using the Advantage 2 PCR™ Enzyme System (CLONTECH) and SMART PCR™ primer. The reaction mix was prepared as follows: 1× Buffer (40 mM Tricine-KOH (pH 9.2), 15 mM KOAc, 3.5 mM Mg(OAc)₂, 3.75 μg/ml BSA, 0.005% Tween-20®, 0.005% Nonidet-P40®); 0.5 mM dNTP, 0.5 mM SMART PCR primer, and 1×Advantage 2 Polymerase. An MJR PCR machine was used for amplification with the following parameters: 95° C. for 1 min.; (95° C. for 15 sec., 65° C. for 30 sec.)-18-26 cycles, 68° C. for 6 min. The number of cycles was optimized individually for each neurosphere.

The use of these procedures streamlines the RT-PCR assay by eliminating time-consuming procedures involved in RNA isolation. All manipulations were carried out in one tube, and amplified cDNA was produced from all RNA available from small numbers of cells. Moreover, the RNA had a high degree of purity. It was possible to detect mRNA transcripts for various genes from neurospheres, or even single cells within neurospheres, using extremely small quantities of a sonicate.

5.2.4. Subtractive Library Preparation

Two cDNA libraries were used in forward and reverse subtractions. For forward subtraction, the first library was a tester and the second one was a driver. For reverse subtraction, the second cDNA library was a tester, and the first one was a driver. The tester and driver cDNAs were digested with RsaI, a four base cutting restriction enzyme that yields blunt ends. The tester cDNA was then subdivided into two equal aliquots and each aliquot ligated with different cDNA adapters. The adapters have stretches of identical sequence to allow annealing of the PCR™ primer once the recessed end has been filled in.

Two hybridizations were then performed. In the first, an excess of driver was added to each sample of tester. The samples were then heat-denatured and allowed to anneal. During the second hybridization, the two primary hybridization samples were mixed together without denaturing, and freshly denatured driver cDNA is added. After filling in the ends by DNA polymerase, the differentially expressed tester sequences had different annealing sites for the nested primers on their 5′ and 3′ ends. The entire population of molecules was then subjected to PCR™ to amplify the desired, differentially expressed sequences. Next, a secondary PCR™ amplification was performed using nested primers to further reduce any background PCR™ products, and enrich the population for differentially expressed sequences.

Before preparation of a subtractive library, a panel of full-length cDNA libraries was screened for the expression of a representative set of housekeeping, cell phenotype, and developmental genes, and arranged according to the degree of “maturity” of neurospheres (see Table 3 and Table 4). As depicted in Table 4, two neighboring libraries were chosen for subtraction. These libraries were amplified using the SMART approach, and this produced 100 μl of template for LD amplification with ten tubes, using 24 cycles for each neurosphere. Two tubes were kept for further applications, and 8 tubes used for subtraction. Tester and driver cDNA was ethanol-precipitated using 1/10 volume sodium acetate and 2.5 volume ethanol plus 20 μg DNASE 1 treated tRNA (Gibco BRL). The template was concentrated by purification using Amicon-10 concentrators according to the manufacturer's directions. cDNA was subjected to overnight digestion by 60 units of RsaI (New England Biolabs, Beverly, Mass.) at 37° C. The digestion mix was purified and concentrated in an Amicon-10 concentrator. The tester cDNA was subdivided in two aliquots and ligated with different adapters during 20 hr at 16° C. using 2000 units of ligase (New England Biolabs) in 10 μl volumes. The samples were heated at 72° C. for 5 min. to inactivate the ligase. The first hybridization was made in 4 μl volumes using 40×excess of driver. The samples were overlaid with 1 drop of mineral oil and incubated in a thermal cycler at 98° C. for 1.5 min., and then at 68° C. for 10 hr.

For the second hybridization, two samples with different adapters from the first hybridization were mixed together and fresh 10×excess of driver cDNA was added. The reaction tube was incubated at 68° C. overnight, and then kept at −20° C. For PCR™ amplification, the Advantage 2 PCR™ Enzyme System (CLONTECH) was used. The template was diluted 20-fold and 1 μl used for amplification with PCR™ Primer 1 (10 μM). Thirty cycles of thermal cycling were then performed.

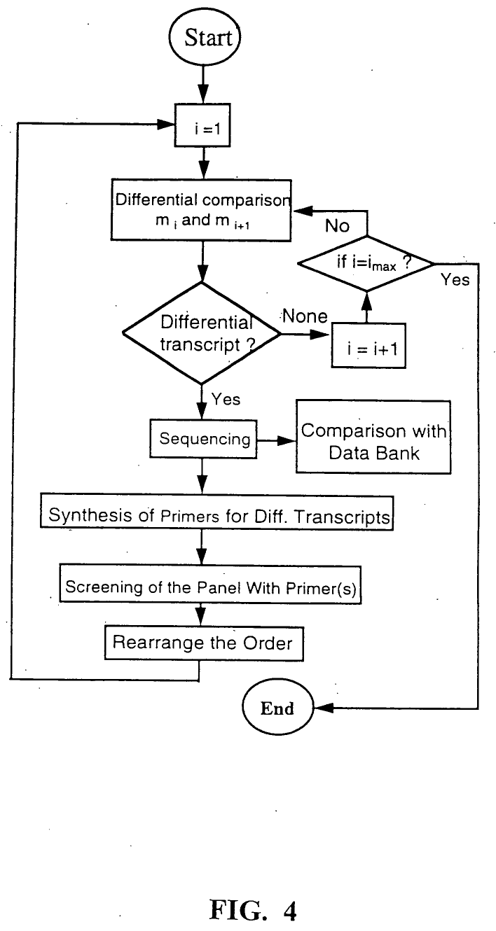

For the second amplification, 1 μl of template from the first LD PCR™ was used with two primers: nested PCR™ primer 1 (10 μM) and nested PCR™ primer 2R (10 μM) for 15 cycles. The product was inserted into a T/A cloning vector, bacteria were transformed, and 96 positive clones were selected for further analysis. Clones were tested for the presence of cDNA inserts as differential transcripts and sequenced. The results of sequencing were compared to known sequences present in GeneBank, and, as shown at the bottom of the iterative algorithm (FIG. 4), all cDNA from a given panel was screened. Additional iterations were performed, see FIG. 4.

The detection of mRNA expression of particular glial and neuronal phenotype markers, GFAP, nestin, and tenascin in preliminary studies (Kukekov et al., 1997; Steindler et al., 1998) employed “nested” primers with Touch Down (TD)-PCR Nested primers were used to eliminate nonspecific amplification. Primers were created using the program Oligo 5.1, and were obtained from Gibco, Life Technologies (Gaithersburg, Md.).

PCR products were then analyzed. 2% agarose gels containing ethidium bromide were used to visualize gene transcripts from individual versus adult brain microclones for a variety of cell phenotypes and developmental genes. After cDNA library production, cDNAs from individual microclones were stored frozen for subsequent analysis. They may be used to develop microarrays following plating on glass or nylon substrates.

### 5.3.0 Example 3

### Microarray Panels

Microarrays can be made using cDNA fragments, and these fragments allow the screening of numerous genes (potentially thousands per microclone) that define cellular phenotype as well as developmental and differentiation status of an individual microclone.

Brain tissue was dissociated and plated to methylcellulose as described (Kukekov et al., 1997; Kukekov et al., 1999). cDNA libraries were prepared from neurospheres and tested according to the iterative algorithm described below. This algorithm allows cDNA libraries from individual neurospheres to be arranged into panels according to the state of maturation of each neurosphere.

Comparisons of neighboring pairs of cDNA libraries resulted in the recognition of differential transcripts which reflect differences in gene expression between neighboring neurospheres. These transcripts, containing representative cDNA fragments, can then be plated onto different microchips as DNA microarrays. These microarrays will be created to reflect the sequential expression of different genes during neural development The inventors contemplate that the microarrays can be used for screening of any cDNA library, following hybridization on the microarray.

Following microarray production, gene transcripts will be plated in random patterns, and repeatedly screened using an algorithm to order arrays and determine gene expression in a functionally significant manner. This defined patterns of developmental and cell phenotype gene expression within a single, developmentally-distinct microclone. The inventors contemplate that patterns of gene expression within individual and different microclones can be confirmed and extended to analyses on microclones themselves following the generation of oligonucleotide- or riboprobes and their application in in situ hybridization or RT-PCR in situ hybridization.

FIG. 2 shows the flowchart for the method and application of cDNA microarray pools for neural gene discovery. Brain tissue was dissociated and plated to methylcellulose as described above. cDNA pools were prepared from microclones and tested according to the iterative algorithm shown in FIG. 4. This algorithm is used to provide information needed to arrange cDNA pools from individual microclones into panels according to the state of maturation of each microclone. Comparison of neighboring pairs of cDNA pools from each panel results in the recognition of differential transcripts which reflect the difference in gene expression between neighboring microspheres. These transcripts will contain representative cDNA fragments when plated onto different microchips as DNA microarrays. These microarrays will be created to reflect the sequential expression of different genes during neural development. They can be used for screening of any cDNA library, following hybridization on the microarray.

5.3.1 Temporal Microchip Array

Microchip array approaches can be produced from ongoing screening of cDNA libraries. However, the arrays created from panels of microclonal cDNA pools of the present invention are quite different from those previously described. The panels of cDNA libraries derived from brain cell microclones contain not only all genes participating in neurogenesis, but also a temporal patterning profile of gene expression. Other described arrays are one-dimensional; they do not contain this temporal information. Thus, the difference between the existing arrays and the arrays provided from the present invention is similar to the difference between a single snap-shot and a movie. The panels and methods described provide sequential information on gene expression. High throughput analysis can be applied to the sequence of genes present in any given array as well as to temporal expression patterns of genes.

5.3.2 Gene Transcript Comparison From Brain Microclones

Microarrays can be made from various brain microclones. FIG. 3 shows a representative sample of the RT-PCR products generated from single (FIGs. A-F) versus multiple (Figs. G-J) microclones. The photomicrograph of an agarose gel shows the presence of gene transcripts for various neuronal, glial, and developmental markers expressed in individual and populations of brain microclones. On either side of lanes A-J FIG. 3) are DNA ladders. Transcripts are as follows: A: β-actin; B: GFAP; C: tenascin (nested primer); D: nestin (nested primer); E: neurofilament-m; F: HuD (a neuron and neuron progenitor marker; G: p-actin; H: tenascin; I: GFAP (nested primer), and J: nestin (nested primer).

Single microclones express combinations of genes that are also expressed by populations of microclones. Previous studies have reported gene expression from populations of neurospheres; now, for the first time, the ability to detect specific gene expression in individual microclones has been demonstrated.

In addition to comparing individual versus populations of microclones, two or more cDNA libraries from different individual microclones can also be compared. This type of comparison leads to identification of differential transcripts among these microclones, such as full length cDNA, or cDNA fragments which are products of differentially expressed genes.

### 5.4.1 Example 4

### Temporal Arrangement of cDNA Pools

The present invention utilizes suppressive substractive hybridization (SSH) to confirm distinct gene expression patterns across clones and to identify novel transcripts. Gene expression that varies across microclones can be used to determine temporal variation in gene expression. Other differential methods including enzymatic degrading subtraction (EDS, Zeng et al., 1994) and representative differential analysis (RDA) are also useful in differential gene expression studies of microarrays. By employing the disclosed panel arrays and methods, it is possible to characterize changes in gene expression within and across different populations of brain cells.

The use of microarrays combined with an iterative process (see FIG. 4) affords a quick and reliable method for the concomitant screening of thousands of gene fragments.

5.4.2 Iterative Ordering of Gene Expression

The schematic diagram of the algorithm used to create an orderly arrangement of cDNA pools is shown in FIG. 4. Application of this algorithm begins with a panel of cDNA pools that are not arranged in any orderly, functional manner (“i=1”). The first step is to compare the first pair of pools using differential analysis to determine the presence or absence of distinct transcripts. If no differential transcript is found, the comparison of the next pair of pools is started. For example, the second pool is compared with the third pool by returning to the beginning of the algorithm replacing “i=1” with “i=i+1,” leading to “mᵢ” and “mᵢ₊₁” where m is a particular pool.

If differential transcripts are found, they are then sequenced and the sequences are compared with published sequences within a Data Bank. If these sequences belong to known genes, they are thus named from the Data Bank. If they are novel sequences, then primers are made to these sequences, and a screening is made on this panel using these primers to determine in which pools these genes are expressed. Then, the order of pools within a panel is arranged according to the earliest appearance of a particular gene transcript using a cluster analysis algorithm (CLUSTER, SAS Multivariate Statistics Package). Once rearranged within this sequential/temporal order of expression, pools can again be subjected to the same algorithm (“start”). This algorithm is repeated, iteratively, until “i” equal the number of pools in a particular panel (“i=iₘₐₓ”), and the “end” of the screen. This yields are differential transcripts present during any given stage of neural development from the earliest stages of neuro-morphogenesis until the most mature. At different stages of neurogenesis (e.g., a progression from undifferentiated stem/progenitor cells to terminally differential cells), difference cascades (clusters) of genes are expressed.

In FIG. 4, stage 1 (“i=1”) represents the earliest stem/progenitor cells, and intermediate stages represent progenitors at different stages of their maturation. Stage “n” (“i=iₘₐₓ”) represents terminally differentiated cell morphotypes, including neurons or glia.

Each neurosphere is presumed to arise from a single stem cell (or SFC) (Scheffler et al, 1999). During stem cell proliferation, they undergo asymmetric divisions resulting in the generation of one copy of the “mother” cell as well as a less pluripotent progenitor cell. Progenitor cells produce progeny which are more restricted than their “parents” because of cell-cell signaling and extrinsic signals from the growth media This process is repeated several times, each time producing more “mature” morphotypes and eventually terminally differentiated cells. Therefore, each neurosphere consists of the mixture of cells ranging from stem cells—to more restricted progenitors at the different stages of development—and, in some cases, terminally differentiated cells. FIG. 4 is a schematization of the pattern of gene expression during the process of maturation of a neurosphere.

In FIG. 4, stage 1 represents gene expression in a neurosphere at the earliest stage of development, again consisting only of stem cells. Stages 2 (the second iteration after “Rearrange the order”, where one goes back to “i=1”) and on representative gene expression patterns during different stages of neurosphere maturation. According to this model, due to asynchronous proliferation and differentiation of cells, the population of neurospheres must include diverse types of neurospheres in the range from the most immature to terminally differentiated cells (e.g., neurons and glia). To test this hypothesis, 30 samples of human neurospheres were randomly selected from a single population, cDNA libraries were prepared from each neurosphere, and each analyzed for the expression of a number of differential markers by RT-PCR™. The result of the analysis is presented in Table 3.

TABLE 3
Variability in Gene Expression Within a Population of Neurospheres
CLONE NUMBER

| | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| b-actin | xxx | | xxx | | | | | xxx | | xxx | xxx | xxx | xxx | xxx | xxx | xxx |
| b-2-micro | | | | | | | | xxx | xxx | xxx | | xxx | xxx | xxx | xxx | xxx |
| Nestin | | | | | | | | | | | | | | | xxx | |
| NF-M | | | xxx | | | | | | | | xxx | | | | xxx | xxx |
| NSE | | | | | | | | | x | | xxx | x | xxx | xxx | xxx | xxx |
| MAP2 | | | x | | | x | | | | x | | | x | | x | x |
| GFAP | | | | | | | | | | | | x | | | x | xxx |
| Tenascin | | | | | | x | | xxx | | x | x | xxx | | x | | |
| Pax-6 | | x | | | | | | | x | | | xxx | | xxx | | xxx |

| | 17 | 18 | 19 | 20 | 21 | 22 | 23 | 24 | 25 | 26 | 27 | 28 | 29 | 30 |
|---|---|---|---|---|---|---|---|---|---|---|---|---|---|---|
| b-actin | xxx | xxx | xxx | xxx | xxx | xxx | xxx | xxx | xxx | xxx | xxx | xxx | xxx | xxx |
| b-2-micro | xxx | xxx | xxx | xxx | xxx | xxx | xxx | xxx | xxx | xxx | xxx | xxx | xxx | xxx |
| Nestin | x | xxx | xxx | | | x | x | x | | | x | xxx | xxx | x |
| NF-M | xxx | xxx | xxx | xxx | | xxx | | xxx | | | | xxx | | xxx |
| NSE | xxx | xxx | xxx | xxx | xxx | | xxx | xxx | x | xxx | xxx | xxx | xxx | xxx |
| MAP2 | x | x | x | | xxx | | | xxx | x | x | x | | | x |
| GFAP | xxx | xxx | xxx | x | xxx | | xxx | x | | xxx | xxx | xxx | xxx | xxx |
| Tenascin | x | xxx | x | xxx | xxx | xxx | x | xxx | x | x | xxx | xxx | x | xxx |
| Pax-6 | xxx | x | xxx | xxx | xxx | xxx | xxx | x | xxx | xxx | xxx | xxx | xxx | xxx |

xxx - data after 1^{st} PCR ™ screening
x - data after 2^{nd} PCR ™ screening with nested primers

Table 3 shows the results from screening of 30 human neurosphere cDNA libraries for a representative set of housekeeping, cell phenotype, and developmental genes (9 different genes in this example: b-actin, b-2-microglobulin, housekeeping genes; NSE, neuron specific enolase, a neuron phenotype marker; Pax-6, a paired box gene used as marker of development; tenascin, an extracellular matrix protein expressed during neural development; GFAP, glial fibrillary acidic protein, a cytoskeletal, intermediate filament protein, phenotypic marker of astrocytes; NF-M, neurofilament-M, a cytoskeletal marker of neurons; nestin, an intermediate filament marker of glial and stem/progenitor cells; and MAP2, microtubule associated protein 2, a cytoskeletal marker of neurons). cDNA libraries in this panel are arranged according to the size and presumed maturity of representative neurospheres (from early type I to late type II or type III, as determined using inverted phase microscopy).

The algorithm starts with a panel of cDNA libraries that are not arranged in any orderly, functional manner. Iteration 1: At the first step of the algorithm, cDNA libraries with numbers 1 and 2 are compared for differential transcripts (“i=1”). If no differential transcript is found, the comparison of the next pair of libraries will be started; that is, compare the second with the third, and return to the beginning of the algorithm replacing “i=1” with “i=i+1,” leading to “mᵢ” and “m₁₊₁” where “m” is a particular library. If differential transcripts are found, they are then clones into vectors, bacteria are transformed, purified transcripts are sequenced, and sequences are compared with published sequences in databases EMBL, GenBank PDP and SWISSPROT using the BlastN/X software package. After comparison, if these sequences belong to known genes, they are thus named from the Data Bank. If transcripts are not found in the Data Bank, they are candidates for new gene discovery and can be further studied.

Differential transcripts discovered at this step of the algorithm are used for screening of the cDNA panel by RT-PCR™ with primers synthesized for each transcript or by dot-blotting. According to the results of the screenings, the order of cDNA libraries within the panel will be rearranged by a hierarchical cluster analysis procedure using any known algorithm of cluster analysis. Once rearranged within the sequential/temporal order of expression, the panel can be subjected to Iteration 2 where the same steps will be performed on the rearranged panel. The algorithm will repeat iterations until i=iₘₐₓ, where iₘₐₓ equals the number of cDNA libraries in the panel. The algorithm is designed in such a way that all genes expressed in a representative set of neurospheres will be discovered and all cDNA libraries will be arranged according to their “maturity,” which will clarify the sequence of gene expression during neurosphere differentiation (the neurogenic model).

A panel of cDNA libraries from Table 3 was used as a model to test applicability of cluster analysis for the rearrangement of the panel according to the degree of neurosphere “maturity.” The CLUSTER procedure (SAS Multivariate Statistics Package) was used for hierarchical analysis. The results are presented in Table 4.

TABLE 4
CLONE NUMBER

| | 4 | 5 | 7 | 2 | 6 | 1 | 3 | 8 | 10 | 11 | 13 | 9 | 25 | 14 | 12 | 27 |
|---|---|---|---|---|---|---|---|---|---|---|---|---|---|---|---|---|
| b-actin | | | | | | xxx | xxx | xxx | xxx | xxx | xxx | | xxx | xxx | xxx | xxx |
| b-2-micro | | | | | | | | xxx | xxx | | xxx | xxx | xxx | xxx | xxx | xxx |
| NSE | | | | | | | | | | xxx | xxx | x | x | xxx | x | xxx |
| Pax-6 | | | | x | | | | | | | | x | xxx | xxx | xxx | xxx |
| Tenascin | | | | | x | | | xxx | x | x | | | x | x | xxx | xxx |
| GFAP | | | | | | | | | | | | | | | x | xxx |
| NF-M | | | | | | | xxx | | | xxx | | | | | | |
| Nestin | | | | | | | | | | | | | | | | x |
| MAP2 | | | | | x | | x | | x | | x | | x | | | x |

| | 26 | 23 | 21 | 16 | 20 | 29 | 28 | 22 | 15 | 17 | 18 | 19 | 24 | 30 |
|---|---|---|---|---|---|---|---|---|---|---|---|---|---|---|
| -actin | xxx | xxx | xxx | xxx | xxx | xxx | xxx | xxx | xxx | xxx | xxx | xxx | xxx | xxx |
| -2-micro | xxx | xxx | xxx | xxx | xxx | xxx | xxx | xxx | xxx | xxx | xxx | xxx | xxx | xxx |
| SE | xxx | xxx | xxx | xxx | xxx | xxx | xxx | | xxx | xxx | xxx | xxx | xxx | xxx |
| ax-6 | xxx | xxx | xxx | xxx | xxx | xxx | xx | xxx | | xxx | x | xxx | x | xxx |
| enascin | x | x | xxx | | xxx | x | xxx | xxx | | x | xxx | x | xxx | xxx |
| FAP | xxx | xxx | xxx | xxx | x | xxx | xxx | | x | xxx | xxx | xxx | x | xxx |
| F-M | | | | xxx | xxx | | xxx | xxx | xxx | xxx | xxx | xxx | xxx | xxx |
| estin | | x | | | | xxx | xxx | x | xxx | x | xxx | xxx | x | x |
| AP2 | x | | | x | | | | | x | x | x | x | xxx | x |

xxx - data after 1^{st} PCR ™ screening
x - data after 2^{nd} PCR ™ screening with nested primers

Table 4 shows the same neurosphere panel as shown in Table 3, rearranged according to the appearance of the 9 different genes. The diversity of neurospheres present in this panel is reflected by the variance in transcript expression from the left side of the table, where there are examples of neurospheres with none of the 9 genes expressed, to the right side of the table where there are examples of neurospheres with all 9 genes expressed. It is generally assumed, from experimental data that neuronal genes are turned on before glial genes during embyrogenesis (Levitt et al., 1981; Jacobson, 1978). The table demonstrates the expression of glial markers such as glial fibrillary acidic protein, GFAP) or neural markers such as neural filaments and microtublue associated protein 2 (NF-M and MAP-2). The spectrum of gene expression during neural development is mirrored in this particular panel by the appearance of differential gene expression between the putative most immature and most differentiated neurosphere.

### 5.5.0 Example 5

### Differential Gene Expression in Individual Clones

Individual clones derived from different human brain progenitor cells were prepared. The results of screening of a human microclonal cDNA panel for a representative set of housekeeping, cell phenotype, and developmental genes are shown in Tables 3 and 4. In this example, nine different genes are screened including β-actin, β-2-microglobulin, housekeeping genes, NSE (neuron specific enolase, a neuron phenotype marker), PAX-6 (a paired box gene used as a marker of development), tenascin (an extracellular matrix protein expressed during neural development), GFAP (glial fibrillary acidic protein, a cytoskeletal, intermediate filament phenotypic marker of astrocytes), NF-M (neurofilament-M, a cytoskeletal marker of neurons), nestin (an intermediate filament marker of glial and precursor cells), and MAP2 (microtubule associated protein 2, a cytoskeletal phenotypic marker of neurons).

In this example, 30 microclones were used for preparation of cDNA pools. Pools in this panel were arranged according to the size and phase darkness or brightness of the microclones as determined by inverted phase microscopy.

5.5.1 Ordering of Microclonal cDNA Pools

The microclonal panels shown in Table 4 were rearranged according to the appearance of the nine different genes. The diversity of microclones present in this panel is reflected by the variance in transcript expression from the left side of Table 4 where there are examples of microclones with none of the nine genes expressed, to the right side of Table 4 where there are examples of microclones with all nine genes expressed. The general assumption from independent experimental data is that neuronal genes are turned on before glial genes during embryogenesis (Levitt et al., 1981; Jacobson, 1978). Table 4 confirms this assumption by demonstrating the expression of the neuronal marker neuron specific enolase (NSE). The spectrum of gene expression during neural development is mirrored in this particular panel by the appearance of differential gene expression between the earliest and latest microclones.

### 5.6.0 Example 6

### Expression of Developmental and Apoptotic Genes in Clonal Populations

Gene expression profiles of clonal populations of normal human stem/progenitor cells and tumor cells were compared. Primitive stem or early progenitor cells are able to initiate hematological and lymphoproliferative neoplasia Brain tumors are also initiated by an event that involves these precursor cells.

Stem and tumor cells were isolated as individual microclones. cDNA was produced from these individual clones as well as from clone mixtures. RT-PCR was used to compare the expression of genes associated with early brain development and apoptosis.

In addition to distinguishing the origin of tumors or tumor cells, knowledge of the temporal gene expression pattern in tumors is useful in the diagnosis, prognosis and treatment strategy of patients from which these tumors are derived. For example, cDNA from microclones derived from tumor cells at various stages leads to the temporal ordering of gene expression as a function of these tumor stages. Thus, when the microclone derived from a specific tumor is analyzed for specific gene expression, the stage of development of this tumor is determined Knowledge of the stage of tumor development (i.e., early or late, for example) helps in determining the prognosis and potential treatment protocol of the patient from which the tumor is derived.

Comparing the cDNA libraries from isolated tumor microclones is useful for identifying genes expressed during the process of tumorgenesis, as well as new anti-tumor drug discovery. Furthermore, the use of microclones derived from tumor cells leads to new approaches to tumor classification. The dedifferentiation disembryoplastic development of any cell cloned is a continuum as genes are turned on and off distinguishing stages of that cell's development. Thus, tumors can be defined by their genetic profile rather than their phenotype or microscopic profile.

Populations from different primary gliomas showed individual profiles of gene expression similar to those of normal human brain stem and progenitor cells. Double immunostaining of glial tumor clones plated on polyornithine/laminim-coated coverslips revealed both neuronal (β-III tubulin) and glial (GFAP) lineages confirming a diversity of morphotypes present within individual clones. These data show that primitive stem or progenitor cells of the human brain can be associated with glioma neoplastic transformation.

### 5.7.0 Example 7

### In Situ Hybridization Using Transcripts From Individual Microclones

cDNA libraries can be obtained from individual or populations of microclones. RT-PCR is performed to generate complementary transcripts. These transcripts may then be used to go back to a tissue or tissue fragment to localize expression of a specific gene.

For example, cDNA libraries can be made from brain cell microclones. RT-PCR is performed on this library to generate a complementary transcript for a specific gene. This complementary transcript is then hybridized to brain tissue or brain tissue fragments in order to localize expression to discrete areas of the brain. In combination with other methods such as immunolabeling, the transcript may be used to localize expression of that gene to a particular population of cells. This method leads to the identification of specific brain cells within the brain tissue in which the specific transcript isolated from that microclonal uncloned cDNA library is expressed.

### 5.8.0 Example 8

### Isolation of New Genes

Clones #9 and #25. (Table 3) were subjected to the subtractive procedure previously described. The product of subtractive hybridization (using SSH) was inserted into a T/A cloning vector; bacteria were transformed using electroporation, and more than 100 clones were obtained for further analysis. 96 of these clones were selected for detailed analysis with insert amplification using PCR for each of the 96 selected clones, and finally, 96-dot cDNA arrays were prepared for further screening.

In order to avoid false positives, a 96-dot cDNA array was hybridized with both forward- and reverse-subtracted probes. Six clones were selected for further detailed analysis. Northern blot analysis is not necessarily performed, since it requires microgram amounts of stem/progenitor cell-specific mRNA. DNA sequence analysis of the fragments was performed, and searches were also made for homology of selected fragments to previously known sequences reported in databases (EMBL, GenBank PDP and SWISS-PROT) using the BlastN/X software package (Table 5).

TABLE 5
Summary of Suppression Subtractive Hybridization (SSH) Fragments

| | Insert | | % of Homology | | |
|---|---|---|---|---|---|
| Clone | Length | | | Pos- | Accession |
| Name | (bp) | BLAST Homology | Identities | itives | Number |
| A4 | 412 | Human cytochrome | 100 | | AF035429 |
| | | oxidase subunit 1 | | | |
| A11 | 439 | Human calcyclin- | 100 | | AF057356 |
| | | binding protein | | | |
| C6 | 204 | No significant | N/A | | N/A |
| C9 | 258 | No significant | N/A | | N/A |
| C10 | 260 | 3′untranslated region | 98 | | U78045 |
| | | of human stromelysin | | | |
| E11 | 270 | Myc-type, ‘helix-loop- | N/A | | N/A |
| | | helix’ dimerization | | | |
| | | domain signature | | | |
| F4 | 268 | 1) Human focal | 34 | 46 | Q14289 |
| | | adhesion kinase 2 | | | |
| | | 2) Homeotic protein | 33 | 42 | P399770 |
| | | spalt-major | | | |
| | | 3) Mouse hypothetical | 32 | 41 | P11260 |
| | | protein ORF-1137 | | | |
| F9 | 480 | Human intercellular | 35 | 48 | P32942 |
| | | adhesion molecule-3 | | | |
| | | precursor | | | |

5.8.1 Clone Description

Clone A4 was shown to be identical to human cytochrome oxidase subunit 1, which is essential for energy conversion in all aerobic organisms.

Clone A11 was shown to be identical to human calcyclin-binding protein (CacyBP), which was identified in human and mouse brains and Ehrlich ascites tumor (EAT) cells and is expressed predominantly there. Because CacyBP, like calcyclin, is present in the brain, the interaction of these two proteins might be involved in calcium signaling pathways in neurol tissue.

Clone C6 had no significant homology to previously sequences reported in databases.

Clone C9 had no significant homology to previously sequences reported in databases.

Clone C10 had strong homology to 3′ untranslated region of stromelysin, human metalloproteinase (MMP) responsible for the breakdown of proteins of connective tissue. Through this action they play an important role in growth, development and tissue repair. Recent studies also suggest that MMPs are utilized in cancer, facilitating both local tumor invasion and metastasis.

Clone E11 did not have any strong homology, but exhibits a Myc-type, ‘helix-loop-helix’ dimerization domain signature. The myc genes are thought to play a role in cellular differentiation and proliferation.

Clone F4 revealed homologies to:

1) Human focal adhesion kinase 2 (FADK 2) (Proline-rich tyrosine kinase 2) (Cell adhesion kinase Beta) (CAK Beta)

Query: 201
KDLPPEQERKRRERTPKNLGNRDEHRTERKRRTPIPQPTHWGPEHSRPRWNMGPPLKTLL 22
KD+ EQER R RTPK L T +P P+ SRP++ PP +T L
KDIAMEQERNARYRTPKIL-----------EPTAFQEPP---PKPSRPKYR--PPPQTNL
Sbjct 730:
687 Query: 21 M
+
Sbjct: 731 L

This protein is involved in calcium induced ion channel regulation, and activation of the MAP kinase signaling pathway. It may represent an important signaling intermediary between neuropeptide-activated receptors or neurotransmitters that increase calcium flux, as well as downstream signals that regulate neuronal activity.

2) Salm drome homeotic protein spalt-major

Query: 195
LPPEQERKRRERTPKNLGNRDEHRTERKRRTPIPQPTHWGPEHSRPRWNMGPPL 34
LP E K + +++HR E RRTP P H P H R PP+
Sbjct: 634
LPLEVRIKEERVEEQEQVKQEDHRIE-PRRTPSPSSEHRSPHHHRHSHMGYPPV 686

This is a transcriptional factor encoded by the spalt major (salm) gene, which is expressed during Drosophila embryogenesis. This protein is found in a broad wedge centered over the decapentaplegic (dpp) stripe, and is one target of Dpp signaling.

3) Mouse hypothetical protein ORF-1137

Query: 183
QERKRRERTPKNLGNRDEHRTERKRRTPIPQPTHWGPEHSRPRWNMGPPLKTLLM 19
Q K + + P N +H +R TP P P H N+ P LKT LM
Sbjct: 22
QMAKGKRKNPTN--RNQDHSPSSERSTPTPP----SPGHPNTTENLDPDLKTFLM 70

Clone F9 was found to be homologous to human intercellular adhesion molecule-3 precursor

Query: 44
EAPTPCLAVSAKTTVGLTEVSLCSCAPSQPLLNGLRV-----GSQFFCGACLEVSGYYLK 208
E T ++ A V +T + + AP QP L G FFC A LE V G +L
Sbjct: 328
EGSTVTVSCMAGARVQVTLDGVPAAAPGQPAQLQLNATESDDGRSFFCSATLEVDGEFLH 387
Query: 209 DFSLIRLPFL 238
S ++L L
Sbjct: 388 RNSSVQLRVL 397

Human intercellular adhesion molecule-3 (ICAM)-3 or CDw50 differentiation antigen is expressed by hematopoietic cells, and not by other cells examined to date. Immunochemical, functional, and protein sequencing studies have shown that this protein presumably plays an important role in the immune response.

This method may be used to perform differential screening of neurospheres at different stages of development/differentiation, and such differential screening can disclose potential differential gene expression between two neurospheres which differentially express unknown as well as known genes. With an initial set of screenings from one set of libraries, from a single subtraction novel genes may be identified that are likely to be important for neurogenesis and neural cell differentiation.

### 6.0 REFERENCES

The following references, to the extent that they provide exemplary procedural or other details supplementary to those set forth herein, are specifically incorporated herein by reference.
U.S. Pat. No. 4,554,101, issued Nov. 19, 1985.
U.S. Pat. No. 4,683,195, issued Jul. 28, 1987.
U.S. Pat. No. 4,683,202, issued Jul. 28, 1987.
U.S. Pat. No. 4,873,192, issued Oct. 10, 1989.
U.S. Pat. No. 4,965,188, issued Oct. 23, 1990.
U.S. Pat. No. 5,176,995, issued Oct. 15, 1991.
U.S. Pat. No. 5,610,288, issued Mar. 11, 1997.
U.S. Pat. No. 5,718,709, issued Feb. 17, 1998.
U.S. Pat. No. 5,739,119, issued Apr. 14, 1998.
U.S. Pat. No. 5,759,829, issued Jun. 2, 1998.
U.S. Pat. No. 5,789,573, issued Aug. 4, 1998.
U.S. Pat. No. 5,801,154, issued Sep. 1, 1998.
European Patent Application Publication No. 75,444.
Adams et al., Nat'l Genet., 4:373-380, 1993.
Allen, J. Comp. Neurol., 22:547-568, 1912.
Altman, J. Comp. Neurol., 136(3):269-293, 1969a.
Altman, J. Comp. Neurol., 137(4):433-457, 1969b.
Altschul et al., “Basic local alignment search tool,” J. Mol. Biol., 215:403-410, 1990.
Arsenijevic and Weiss, J. Neurosci., 18(6):2118-2128, 1998.
Bjornson et al., Science, 283(5401):534-537, 1999.
Calof et al., J. Neurobiol., 36:190-205, 1998.
Chang and Hemmati-Brivanlou, J. Neurobiol., 36:128-151, 1998.
Chen et al., Genomics, 51:313-324, 1998.
Corpet, Nucleic Acids Res., 16:10881-10890, 1988.
Ditachenko et al., Proc. Natl. Acad. Sci. USA, 93:6025-6030, 1996.
Doe, Fuerstenberg and Peng, J. Neurobiol., 36:111-127, 1998.
Eberwine et al., Proc. Nat'l Acad. Sci. USA, 89:3010-3014, 1992.
Eisen et al., Proc. Natl. Acad. Sci. USA, 95(25):14863-14868, 1998.
Eriksson et al., Nat'l Med., 4:1313-1317, 1998.
Ferguson, Nat'l Biotech, 14:1681-1684, 1996.
Gritti et al., Neuron, 16:1091-1100, 1996.
Herington, Biochem. Pharmacol., 35(8):1359-1364, 1986.
Higgins et al., Gene, 73:237-244, 1988.
Higgins et al., CABIOS, 5:151-153, 1989.
Huang et al., Computer Applications in the Biosciences, 8:155-165, 1992.
Hubank and Schatz, Nucleic Acids Res., 22:5640-5648, 1994.
Innis et al., Eds. In: PCR Protocols, A Guide to Methods and Applications, Academic Press, New York, 1990.
Jacobson, In: Developmental Neurobiology, 2^{nd} Edition, Plenum Press, NY, 1978.
Johansson et al., Cell, 96:25-34, 1999.
Keller and Snodgrass, Nat. Med., 5(2):151-152, 1999.
Klebe et al, Biotechniques, 21(6):1094-1100, 1996.
Kuby, Immunology, 2nd Edition, W. H. Freeman & Company, New York, 1994.
Kukekov et al., Exp. Neurol., 156:333-344, 1999.
Kukekov et al., GLIA, 21:399-407, 1997.
Kunkel et al., Methods Enzymol., 154:367-382, 1987.
Kunkel, Proc. Natl. Acad. Sci USA, 82:488492, 1985.
Kyte and Doolittle, J. Mol. Biol., 157:105-132, 1982.
Laywell, Kukekov and Steindler, Exp. Neurol., 156(2):430-433, 1999.
Levitt, et al., J. Neuroscience, 27-39, 1981.
Lin et al., Neurobiol. Dis., 2(2):79-85, 1995.
Ling and Neben, J. Cell Physiol., 171:104-115, 1997.
Lisitsyn et al., Science, 259(5097):946-951, 1993.
Maloy et al., In: Microbial Genetics, 2nd Edition, Jones and Bartlett Publishers, Boston, Mass., 1994.
Mayer-Proschel et al., Neuron, 19(4):773-785, 1997.
McKay, Science, 276:66-71, 1997.
Needleman et al., J. Mol. Biol., 48:443, 1970.
Pearson et al., Proc. Natl. Acad. Sci. USA, 85:2444, 1988.
Person et al., Methods of Molecular Biology, 24:307-331, 1994.
Polymeropoulos et al., Genomics, 12:496-496, 1992.
Prokop and Bajpai, “Recombinant DNA Technology I,” Ann. N.Y. Acad. Sci., Volume 646, 1991.
Ramsay, Natl. Biotech., 16:40-44, 1998.
Reynolds and Weiss, Dev. Biol, 175:1-13, 1996.
Reynolds and Weiss, Science, 225:1707-1710, 1992.
Richards, Kilpatrick and Bartlett, Proc. Natl. Acad. Sci. USA, 89:8591-8595, 1992.
Sambrook et al., In: Molecular Cloning: A Laboratory Manual, 2nd Ed.; Cold Spring Harbor Laboratory Press, Plainview, N.Y., 1989.
Scheffler et al., Trends Neurosci., 22(8):348-357, 1999.
Schena et al., Science, 270:467-470, 1995.
Schena et al., Trends Biotech., 16:301-306, 1998.
Segal, “Biochemical Calculations” 2nd Edition, John Wiley & Sons, New York, 1976.
Shamblott et al., Proc. Natl. Acad. Sci. USA, 95:13726-13731, 1998.
Smith et al., Adv. Appl. Math., 2:482, 1981.
Steindler, Kadrie, Fillmore and Thomas, Prog. Brain Res., 108:349-363, 1996.
Steindler et al., Prog. Brain Res., 117:179-196, 1998.
Stoykova and Gruss, J. Neurosci., 14:1395-412, 1994.
Suslov et al., J. Neurosci. Methods, 96(1):57-61, 2000.
Thomas, Gates and Steindler, GLIA, 17:1-14, 1996.
Thomson et al., Science, 282:1145-1147, 1998.
Van Gelder et al., Proc. Natl. Acad. Sci. USA, 87:1663-1667, 1990.
Walker and Gaastra, Eds., Techniques in Molecular Biology, MacMillan Publishing Company, New York, 1983.
Welford et al., Nucleic Acids Res., 26:3059-3065, 1998.
Zeng et al., Nucleic Acids Res., 22:4381-4385, 1994.

1. A method of making a cDNA library comprising:
culturing at least one cell from a normal or diseased brain under conditions to form at least one single microclone;
disrupting said single microclone to release mRNA;
making cDNA by first strand synthesis of the mRNA; and
amplifying said first strand synthesis cDNA to produce a cDNA library.

2-58. (canceled)

59. The method of claim 1, wherein the microclone is a neurosphere immunonegative for GFAP, nestin, β-III tubulin and L1.

60. The method of claim 1, wherein the microclone is a neurosphere immunopositive for nestin but immunonegative for GFAP and β-III tubulin.

61. The method of claim 1, wherein the microclone is a neurosphere that readily attaches to plastic and laminin substrates.

62. The method of claim 61, wherein the neurosphere is immunopositive for nestin, GFAP, and β-III tubulin.

63. The method of claim 1, wherein the microclone is derived from a brain tumor cell.

64. The method of claim 1, wherein the microclone is derived from a hematopoietic cell.

65. The method of claim 1, wherein the brain is from a human.

66. The method of claim 65, wherein the human is an adult.

67. The method of claim 65, wherein the microclone is immunopositive for tenascin.

68. The method of claim 1, wherein the microclone is derived from postmortem brain tissue.

69. The method of claim 1, wherein the microclone is derived from cells of the temporal lobe or subependymal zone of the brain.

70. The method of claim 1, wherein the brain is from a human with a disease selected from the group consisting of brain cancer, Alzheimer's disease, Parkinson's disease and Huntington's disease.

71. A method for isolating gene transcripts that are differentially expressed during a selected stage of a developmental sequence, comprising;
(a) culturing a stem cell under conditions to produce a first microclone comprising cells at a first stage of development;
(b) disrupting said first microclone, amplifying RNA of said cells of said first microclone and preparing a first microclonal cDNA library from said amplified RNA;
(c) preparing a second microclonal cDNA library from RNA of cells of a second microclone at a second stage of development;
(d) comparing RNA transcripts from said first and said second cDNA libraries to identify at least one transcript differentially expressed in one of said first or second cDNA libraries; and
(e) correlating expression of said at least one identified transcript with a selected stage of development.

72. The method of claim 71, wherein the developmental sequence is neurogenesis.

73. The method of claim 71, wherein the developmental sequence is oncogenesis.

74. The method of claim 71, wherein the developmental sequence is hematopoeisis.

75. The method of claim 71, wherein said first microclone and said second microclone are neurospheres at different stages of development.

76. The method of claim 71, wherein the stem cell is derived from a normal or diseased human brain.

77. The method of claim 71, wherein at least one of said first and second cDNA libraries is characterized by absence of transcripts for Pax-6.


SEQUENCE LISTING (11 sequences)

SEQ ID NO: 1 - protein, 60 aa, Homo sapiens [sequence omitted]

SEQ ID NO: 2 - protein, 4 aa, Homo sapiens
EQER

SEQ ID NO: 3 - protein, 4 aa, Homo sapiens
RTPK

SEQ ID NO: 4 - protein, 44 aa, Homo sapiens
KDIAMEQERNARYRTPKILEPTAFQEPPPKPSRPKYRPPPQTNL

SEQ ID NO: 5 - protein, 54 aa, Homo sapiens
LPPEQERKRRERTPKNLGNRDEHRTERKRRTPIPQPTHWGPEHSRPRWNMGPPL

SEQ ID NO: 6 - protein, 4 aa, Homo sapiens
RRTP

SEQ ID NO: 7 - protein, 53 aa, Drosophila melanogaster
LPLEVRIKEERVEEQEQVKQEDHRIEPRRTPSPSSEHRSPHHHRHSHMGYPPV

SEQ ID NO: 8 - protein, 55 aa, Homo sapiens
QERKRRERTPKNLGNRDEHRTERKRRTPIPQPTHWGPEHSRPRWNMGPPLKTLLM

SEQ ID NO: 9 - protein, 49 aa, Mus musculus
QMAKGKRKNPTNRNQDHSPSSERSTPTPPSPGHPNTTENLDPDLKTFLM

SEQ ID NO: 10 - protein, 65 aa, Homo sapiens
EAPTPCLAVSAKTTVGLTEVSLCSCAPSQPLLNGLRVGSQFFCGACLEVSGYYLKDFSLIRLPFL

SEQ ID NO: 11 - protein, 70 aa, Homo sapiens
EGSTVTVSCMAGARVQVTLDGVPAAAPGQPAQLQLNATESDDGRSFFCSATLEVDGEFLHRNSSVQLRVL